(19) United States
(12) Patent Application Publication
(10) Pub. No.: US 2025/0387431 A1
(43) Pub. Date: Dec. 25, 2025

(54) TREATING CANCER WITH CHIMERIC ANTIGEN RECEPTORS THAT BIND TO TRAILSHORT POLYPEPTIDES

(71) Applicant: Mayo Foundation for Medical Education and Research, Rochester, MN (US)

(72) Inventors: Saad J. Kenderian, Rochester, MN (US); Andrew D. Badley, Rochester, MN (US); Ismail Can, Rochester, MN (US); Sekar Natesampillai, Rochester, MN (US)

(21) Appl. No.: 18/878,277

(22) PCT Filed: Jun. 23, 2023
(86) PCT No.: PCT/US2023/026057

§ 371 Date: Dec. 23, 2024

Related U.S. Application Data
(60) Provisional application No. 63/355,394, filed on Jun. 24, 2022.

(51) Int. Cl. A61K 35/17; A61K 40/11; A61K 40/31; A61K 40/42; A61P 35/00; C07K 14/705; C07K 14/725; C07K 16/28; C12N 5/0783

(52) U.S. Cl. CPC A61K 35/17; A61K 40/11; A61K 40/31; A61K 40/4202; A61P 35/00; C07K 14/7051; C07K 14/70517; C07K 14/70578; C07K 16/2803; C07K 16/2875; C12N 5/0636; A61K 2239/13; A61K 2239/17; A61K 2239/21; C07K 2317/53; C07K 2317/622; C07K 2319/02; C07K 2319/03; C12N 2510/00

(57) ABSTRACT

This document provides methods and materials for binding a chimeric antigen receptor (CAR) to a TRAILshort polypeptide. For example, a CAR that binds to a TRAILshort polypeptide and methods and materials for using cells expressing such a CAR to treat a mammal (e.g., a human) having cancer are provided. Further, a chimeric antigen receptor comprising an antigen binding domain, a hinge, a transmembrane domain, and one or more signaling domains.

### CROSS-REFERENCE TO RELATED APPLICATIONS

This application claims priority from U.S. Provisional Application Ser. No. 63/355,394, filed Jun. 24, 2022. The disclosure of the prior application is considered part of (and is incorporated by reference in) the disclosure of this application.

### SEQUENCE LISTING

This application contains a Sequence Listing that has been submitted electronically as an XML file named “07039-2108WO1_SL ST26.XML.” The XML file, created on Jun. 21, 2023, is 148,041 bytes in size. The material in the XML file is hereby incorporated by reference in its entirety.

### BACKGROUND

### 1. Technical Field

This document relates to methods and materials involved in binding a chimeric antigen receptor (CAR) to a TRAILshort polypeptide, which is a splice variant of a TNF related apoptosis inducing ligand (TRAIL) polypeptide. For example, this document provides CARs that bind to TRAILshort polypeptides, and methods and materials for using such CARs to treat cancer and infections (e.g., chronic infections). This document also provides cells (e.g., host cells) designed to express one or more CARs having the ability to bind to a TRAILshort polypeptide, and methods and materials for using such cells to treat cancer and infections.

### 2. Background Information

TRAIL is a member of the tumor necrosis factor (TNF) superfamily of death-inducing ligands whose members include Fas ligand and TNF. Ligation of TRAIL to its cognate receptors can cause cell death by apoptosis or may cause NF-κB activation (Hu et al., J. Biol. Chem., 274:30603-10 (1999)). TRAIL is widely expressed on multiple cell lineages and has potent toxicity for many tumors and virally infected cells, while sparing most healthy cells (Held et al., Drug Resist. Updat., 4:243-52 (2001); and Baetu and Hiscott, Cytokine Growth Factor, 13:199-207 (2002)). TRAIL mediates cell death via binding of one of five TRAIL receptors (e.g., TRAIL-R1, -R2, -R3, -R4, and osteoprotegerin (OPG)). TRAILshort is a splice variant of TRAIL that is capable of blocking TRAIL mediated cell death.

### SUMMARY

This document provides methods and materials involved in binding a CAR to a TRAILshort polypeptide. TRAILshort is a splice variant of TRAIL, and is capable of binding to TRAIL-R1 (“R1”) and/or TRAIL-R2 (“R2”). When bound to R1 and/or R2, TRAILshort prevents full length TRAIL from inducing cell death, as described elsewhere (Schnepple et al., 2011 J Biol Chem 286, 35742-35754). As described herein, T cells expressing a CAR that can specifically bind TRAILshort polypeptide (TsCAR T cells) exhibit potent antitumor efficacy. T cell activation (e.g., with PMA pre-treatment) can be used to increase TRAILshort expression and can increase cytotoxicity of TsCAR T cells. In some cases, T cells expressing two CARS (e.g., a CAR that can specifically bind TRAILshort polypeptide and a CAR that can specifically bind CD19) can be used to increase the anti-tumor activity.

In some embodiments, this document provides CARs that bind to a TRAILshort polypeptide, and methods and materials for using one or more such CARs to treat a mammal (e.g., a human) having cancer, e.g., a TRAILshort+ cancer.

This document also provides cells (e.g., host cells) designed to express one or more CARs having the ability to bind to a TRAILshort polypeptide, and methods and materials for using such cells to treat cancer, e.g., a TRAILshort+ cancer.

As described herein, one or more CARs can be designed to have the ability to bind to a TRAILshort polypeptide. For example, a CAR provided herein can have the ability to bind to a polypeptide comprising, consisting essentially of, or consisting of the amino acid sequence of a human TRAILshort polypeptide as set forth in SEQ ID NO:34 or 35 (see, e.g., FIG. 1).

In some cases, two sets of three CDRs of an antigen binding fragment provided herein (e.g., SEQ ID NOs: 1-3 and SEQ ID NO:9, the amino acid sequence GAS, and SEQ ID NO: 10) can be engineered into a CAR to create CAR⁺ cells (e.g., CAR⁺ T cells, CAR⁺ stem cells such as CAR⁺ induced pluripotent stem cells, or CAR⁺ natural killer (NK) cells) having the ability to target TRAILshort⁺ cells (e.g., TRAILshort⁺ tumor cells),

As also described herein, cells (e.g., host cells) can be designed to express one or more CARs having the ability to bind to a TRAILshort polypeptide. For example, cells such as T cells (e.g., CTLs), stem cells (e.g., induced pluripotent stem cells), or NK cells can be engineered to express one or more CARs having the ability to bind to a TRAILshort polypeptide. Such cells (e.g., TRAILshort-specific CAR⁺ T cells or NK cells) can be used to treat cancer (e.g., a TRAILshort⁺ cancer) or an infection (e.g., a chronic infection) in a mammal. For example, a mammal (e.g., a human) having an infection can be administered a composition comprising one or more cells expressing CARs described herein to reduce the number of infected cells within the mammal. For example, a mammal (e.g., a human) having a TRAILshort+ cancer can be administered a composition comprising one or more cells expressing CARs described herein to reduce the number of cancer cells within the mammal and/or to increase the survival duration of the mammal from cancer.

In one aspect, this document features a chimeric antigen receptor that includes an antigen binding domain, a hinge, a transmembrane domain, and one or more signaling domains, wherein the antigen binding domain comprises a heavy chain variable domain or region comprising the amino acid sequences set forth in SEQ ID NO:1 (or SEQ ID NO: 1 with one, two, or three amino acid additions, deletions, or substitutions), SEQ ID NO: 2 (or SEQ ID NO:2 with one, two, or three amino acid additions, deletions, or substitutions), and SEQ ID NO:3 (or SEQ ID NO:3 with one amino acid addition, deletion, or substitution), and a light chain variable domain or region comprising the amino acid sequence set forth in SEQ ID NO:9 (or SEQ ID NO:9 with one, two, or three amino acid additions, deletions, or substitutions), the amino acid sequence GAS (or GAS with one amino acid addition, deletion, or substitution), and the amino acid sequence set forth in SEQ ID NO:10 (or SEQ ID NO:10 with one, two, or three amino acid additions, deletions, or substitutions). The antigen binding domain can have the ability to bind to a human TRAILshort polypeptide (e.g., the amino acid sequence set forth in SEQ ID NO: 35). The antigen binding domain can include an scFv, e.g., an scFv having the ability to bind to a TRAILshort polypeptide.

In some embodiments, the heavy chain variable domain or region can include an amino acid sequence having at least 90 percent identity to the amino acid sequence set forth in SEQ ID NO:8.

In some embodiments, the light chain variable domain or region can include an amino acid sequence having at least 90 percent identity to the amino acid sequence set forth in SEQ ID NO:15.

In some embodiments, the hinge includes a hinge set forth in FIG. 7A. For example, the hinge can be a CD8 hinge or a CD28 hinge.

In some embodiments, the transmembrane domain includes a transmembrane domain set forth in FIG. 7C. For example, the transmembrane domain can be a CD8 transmembrane domain or a CD28 transmembrane domain.

In some embodiments, the one or more signaling domains are selected from the group of signaling domains set forth in FIG. 7D. For example, the one or more signaling domains comprises one or more of a 4-1BB intracellular signaling domain, a CD28 intracellular signaling domain, or a CD3ζ intracellular signaling domain.

In some embodiments, the hinge comprises a CD8 hinge, the transmembrane domain comprises a CD8 transmembrane domain, and the one or more signaling domains comprises the 4-1BB intracellular signaling domain and the CD3ζ intracellular signaling domain.

In another aspect, this document features a nucleic acid that includes a nucleic acid sequence encoding a chimeric antigen receptor of any of the embodiments described herein and a host cell that includes such a nucleic acid. The nucleic acid can be a viral vector or a phagemid.

This document also features an isolated population of cells, wherein at least one cell of the population comprises a nucleic acid encoding a chimeric antigen receptor described herein. In some embodiments, at least one cell expresses the nucleic acid and comprises the chimeric antigen receptor on the cell surface. The cell can be a T cell, a stem cell, or an NK cell.

In another aspect, this document features an isolated population of cells, wherein at least one cell of the population includes a nucleic acid encoding a first chimeric antigen receptor and a nucleic acid encoding a second chimeric antigen receptor, wherein the first chimeric antigen receptor is a chimeric antigen receptor described herein (e.g., having the ability to bind to a human TRAILshort polypeptide). In some embodiments, at least one cell expresses the nucleic acid encoding the first chimeric antigen receptor and comprises the first chimeric antigen receptor on the cell surface.

In some embodiments, at least one cell expresses the nucleic acid encoding the second chimeric antigen receptor and comprises the second chimeric antigen receptor on the cell surface. The second chimeric antigen receptor includes an antigen binding domain, a hinge, a transmembrane domain, and one or more signaling domains, wherein the antigen binding domain of the second chimeric antigen receptor has the ability to bind to any one of a human CD19 polypeptide, a human B-cell maturation antigen (BCMA) polypeptide, a human thyroid stimulating hormone receptor (TSHR) polypeptide, a human EPH receptor A3 (EPHA3) polypeptide, a human fibroblast growth factor receptor 2 (FGFR2) polypeptide, a human HER2 polypeptide, a human TROP2 polypeptide, a human NY-ESO polypeptide, a human Mesothelin polypeptide, a human EGFR polypeptide, a human EGFRviii polypeptide, a human IL13Ra2 polypeptide, a human Folate receptor alpha polypeptide, a human Folate receptor Beta polypeptide, a human gut integrin (e.g., ITGB7) polypeptide, a human CD103 polypeptide, a human CD83 polypeptide, a human CD22 polypeptide, a human CD20 polypeptide, a human CD79b polypeptide, a human CD79a polypeptide, a human CD123 polypeptide, a human CD33 polypeptide, a human ILR1a polypeptide, a human CD34 polypeptide, a human CD30 polypeptide, a human CD4 polypeptide, a human CD8 polypeptide, a human T cell receptor alpha polypeptide, a human T cell receptor beta polypeptide, a human CD3 polypeptide, a human CD5 polypeptide, a human CD7 polypeptide, a human gp120 polypeptide, a human galactomannan polypeptide, a human PSMA polypeptide, a human MUC polypeptide, a human PD-1 polypeptide, a human CD80 polypeptide, a human CD86 polypeptide, a human CEA polypeptide, a human GPC3 polypeptide, a human ROR1 polypeptide, a human AFP polypeptide, a human CD138 polypeptide, a human CD38 polypeptide, a human CD44v6 polypeptide, a human CD70 polypeptide, a human CLEC12A (CLL-1) polypeptide, a human CS-1 polypeptide, a human FAP polypeptide, a human GPRC5D polypeptide, a human MUC-1 polypeptide, a human MUC16 polypeptide, or a human NKG2D polypeptide.

For example, second chimeric antigen receptor can contain the CDRs of a FMC63 scFv antibody and bind to a CD19 antigen, can contain the CDRs of a MOR208 scFv and bind to a CD19 antigen, can contain the CDRs of a humanized scFv antibody and bind to a CD19 antigen, can contain the CDRs of a 4G7 scFv antibody and bind to a CD19 antigen, can contain the CDRs of a low affinity scFv antibody and bind to a CD19 antigen, can contain the CDRs of a 5E5 scFv antibody and bind to a MUC-1 antigen, can contain the CDRs of a 4D5 scFv antibody and bind to a HER-2 antigen, can contain the CDRs of a FRP5 scFv antibody and bind to a HER-2 antigen, can contain the CDRs of a M27 scFv antibody and bind to a EGFR antigen, can contain the CDRs of a Cetuximab scFv antibody and bind to a EGFR antigen, can contain the CDRs of a C4 based scFv antibody and bind to a Folate receptor alpha antigen, can contain the CDRs of a MOv19 scFv antibody and bind to a Folate receptor alpha antigen, can contain the CDRs of a SS1 scFv antibody and bind to a Mesothelin antigen, can contain the CDRs of a M clone scFv antibody and bind to a Mesothelin antigen, can contain the CDRs of a Amatuximab scFv antibody and bind to a Mesothelin antigen, can contain the CDRs of a Anetumab scFv antibody and bind to a Mesothelin antigen, can contain the CDRs of a ET1402L1 scFv antibody and bind to a AFP antigen, can contain the CDRs of an Anti-CEA scFv antibody and bind to a CEA antigen, can contain the CDRs of a CEACAM5 scFv antibody and bind to a CEA antigen, can contain the CDRs of a hMN14 scFv antibody and bind to a CEA antigen, can contain the CDRs of a 22172,22176 scFv antibody and bind to a CD123 antigen, can contain the CDRs of a humanized scFv antibody and bind to a CD123 antigen, can contain the CDRs of a humanized scFv antibody and bind to a CD123 antigen, can contain the CDRs of a Tagrazofusp based scFv antibody and bind to a CD123 antigen, can contain the CDRs of a MY96 scFv antibody and bind to a CD33 antigen, can contain the CDRs of a humanized scFv antibody and bind to a CD33 antigen, can contain the CDRs of a humanized scFv antibody and bind to a CLEC12A antigen, can contain the CDRs of a CLL1 scFv antibody and bind to a CLEC12A antigen, can contain the CDRs of a M6E7 scFv antibody and bind to a CLEC12A antigen, can contain the CDRs of a m21C9 scFv antibody and bind to a CLEC12A antigen, can contain the CDRs of a M20B1 scFv antibody and bind to a CLEC12A antigen, can contain the CDRs of a M28H12 scFv antibody and bind to a CLEC12A antigen, can contain the CDRs of a M0971 scFv antibody and bind to a CD22 antigen, can contain the CDRs of a humanized scFv clone antibody and bind to a CD22 antigen, can contain the CDRs of a Inotuzumab based scFv antibody and bind to a CD22 antigen, can contain the CDRs of a Moxetumomab based scFv antibody and bind to a CD22 antigen, can contain the CDRs of a Rituximab scFv antibody and bind to a CD20 antigen, can contain the CDRs of a Leu 16 scFv antibody and bind to a CD20 antigen, can contain the CDRs of a CD20 scFv antibody and bind to a CD20 antigen, can contain the CDRs of a BCMA-02 scFv antibody and bind to a BCMA antigen, can contain the CDRs of a LCAR38 scFv antibody and bind to a BCMA antigen, can contain the CDRs of a BCMA scFv antibody and bind to a BCMA antigen, can contain the CDRs of a biepitopic scFv antibody and bind to a BCMA antigen, can contain the CDRs of a NVS BCMA scFv antibody and bind to a BCMA antigen, can contain the CDRs of a CSIR scFv antibody and bind to a CS-1 antigen, can contain the CDRs of a CS1 scFv antibody and bind to a CS-1 antigen, can contain the CDRs of an Elotuzumab based scFv antibody and bind to a CS-1 antigen, can contain the CDRs of a scFv antibody and bind to a CD138 antigen, can contain the CDRs of a humanized scFv antibody and bind to a CD44v6 antigen, can contain the CDRs of a cMAb U36 scFv antibody and bind to a CD44v6 antigen, can contain the CDRs of a scFv antibody and bind to a NKG2D antigen, can contain the CDRs of a NKG2Dg scFv antibody and bind to a NKG2D antigen, can contain the CDRs of a nanobody CD38cFv antibody and bind to a CD38 antigen, can contain the CDRs of a humanized scFv antibody and bind to a CD38 antigen, can contain the CDRs of a humanized scFv antibody and bind to a GPRC5D antigen, can contain the CDRs of a scFv (L-H) and (H-L) antibody and bind to a CD79b antigen, can contain the CDRs of a M290 scFv antibody and bind to a CD103 antigen, can contain the CDRs of a FAP5 scFv antibody and bind to a FAP antigen, can contain the CDRs of a human CD70 scFv antibody and bind to a CD70 antigen, can contain the CDRs of a 4H11 scFv antibody and bind to a MUC16 antigen, can contain the CDRs of a IL13R scFv antibody and bind to a IL13Ra2 antigen, can contain the CDRs of a Muromonab scFv antibody and bind to a CD3 antigen, can contain the CDRs of a Teplizumab scFv antibody and bind to a CD3 antigen, can contain the CDRs of a Blinatumomab scFv antibody and bind to a CD3 antigen, can contain the CDRs of a Brentuximab scFv antibody and bind to a CD30 antigen, can contain the CDRs of a 4C8 scFv antibody and bind to a CD34 antigen, can contain the CDRs of an Ibalizumab scFv antibody and bind to a CD4 antigen, can contain the CDRs of an Anti-CD5 scFv antibody and bind to a CD5 antigen, can contain the CDRs of a 9F2A11 scFv antibody and bind to a CD5 antigen, can contain the CDRs of an Ab5D7v scFv antibody and bind to a CD5 antigen, can contain the CDRs of a Polatuzumab scFv antibody and bind to a CD7 antigen, can contain the CDRs of an Ab 4450 scFv antibody and bind to a CD79a antigen, can contain the CDRs of an Anti-CD79a scFv antibody and bind to a CD79a antigen, can contain the CDRs of a Crefmirlimab scFv antibody and bind to a CD8 antigen, can contain the CDRs of a Galiximab scFv antibody and bind to a CD80 antigen, can contain the CDRs of a 3C12 scFv antibody and bind to a CD83 antigen, can contain the CDRs of a 32A scFv antibody and bind to a CD86 antigen, can contain the CDRs of an Anti-EGFRVIII scFv antibody and bind to an EGFRviii antigen, can contain the CDRs of an Ifabotuzumab scFv antibody and bind to an EPHA3 antigen, can contain the CDRs of an Aprutumab scFv antibody and bind to a FGFR2 antigen, can contain the CDRs of a Bemarituzumab scFv antibody and bind to a FGFR2 antigen, can contain the CDRs of a M909 scFv antibody and bind to a Folate receptor Beta antigen, can contain the CDRs of an ASO4498 scFv antibody and bind to a Folate receptor Beta antigen, can contain the CDRs of an Antibody #2 scFv antibody and bind to a Folate receptor Beta antigen, can contain the CDRs of an Antibody #3 scFv antibody and bind to a Folate receptor Beta antigen, can contain the CDRs of an EH7 scFv antibody and bind to a galactomannan antigen, can contain the CDRs of a BB10 scFv antibody and bind to a galactomannan antigen, can contain the CDRs of an Elipovimab scFv antibody and bind to a gp120 antigen, can contain the CDRs of a Suvizumab scFv antibody and bind to a gp 120 antigen, can contain the CDRs of a Teropavimab scFv antibody and bind to a gp120 antigen, can contain the CDRs of a Codrituzumab scFv antibody and bind to a GPC3 antigen, can contain the CDRs of an Etrolizumab scFv antibody and bind to a gut integrins antigen, can contain the CDRs of a 10E12 scFv antibody and bind to an ILR1a antigen, can contain the CDRs of a 9E11 scFv antibody and bind to an ILR1a antigen, can contain the CDRs of a 9G5 scFv antibody and bind to an ILR1a antigen, can contain the CDRs of a Cantuzumab scFv antibody and bind to a MUC antigen, can contain the CDRs of a Clivatuzumab scFv antibody and bind to a MUC antigen, can contain the CDRs of a Gatipotuzumab scFv antibody and bind to a MUC antigen, can contain the CDRs of a Sofituzumab scFv antibody and bind to a MUC antigen, can contain the CDRs of a Ubamatamab scFv antibody and bind to a MUC antigen, can contain the CDRs of a 12D7 scFv antibody and bind to a NY-ESO antigen, can contain the CDRs of a T1 scFv antibody and bind to a NY-ESO antigen, can contain the CDRs of a T2 scFv antibody and bind to a NY-ESO antigen, can contain the CDRs of a T3 scFv antibody and bind to a NY-ESO antigen, can contain the CDRs of a Nivolumab scFv antibody and bind to a PD-1 antigen, can contain the CDRs of a Pembrolizumab scFv antibody and bind to a PD-1 antigen, can contain the CDRs of a J591 scFv antibody and bind to a PSMA antigen, can contain the CDRs of a Zilovertamab scFv antibody and bind to a ROR1 antigen, can contain the CDRs of an Anti-TCRa scFv antibody and bind to a T cell receptor alpha antigen, can contain the CDRs of an Anti-TCRBC1 scFv antibody and bind to a T cell receptor beta antigen, can contain the CDRs of a K1-18 scFv antibody and bind to a TSHR antigen, can contain the CDRs of a Sacituzumab scFv antibody and bind to a TROP2 antigen, or can contain the CDRs of a Datopotamab scFv antibody and bind to a TROP2 antigen.

In some cases, the antigen binding domain of the second chimeric antigen receptor can include the CDRs of a FMC63 scFv antibody and bind to a CD19 antigen, comprises the CDRs of a MOR208 scFv and bind to a CD19 antigen, can include the CDRs of a humanized scFv antibody and bind to a CD19 antigen, can include the CDRs of a 4G7 scFv antibody and binds to a CD19 antigen, or can include the CDRs of a low affinity scFv antibody and bind to a CD19 antigen.

The hinge of the second chimeric antigen receptor can be a hinge set forth in FIG. 7A. The transmembrane domain of the second chimeric antigen receptor can be a transmembrane domain set forth in FIG. 7C. The one or more signaling domains of the second chimeric antigen receptor can be selected from the group of signaling domains set forth in FIG. 7D. The cell can be a T cell, a stem cell, or an NK cell.

In another aspect, this document features a method of making a chimeric antigen receptor positive (CAR⁺) cell. The method includes introducing a nucleic acid encoding the CAR into a cell, wherein the cell expresses the CAR, thereby making the CAR⁺ cell. The nucleic acid can be any nucleic acid sequence encoding a chimeric antigen receptor of any of the embodiments described herein. The nucleic acid can be a viral vector and the cell can be infected with the viral vector. The CAR can be expressed on the surface of the cell.

This document also features a composition comprising a population of cells described herein. In some embodiments, at least 50 percent, at least 75 percent, at least 95 percent, at least 99 percent, or 100 percent of the cells express the chimeric antigen receptor. The composition can include a pro-apoptotic compound (e.g., a Bcl-2 inhibitor, an inhibitor of apoptosis (IAP) inhibitor, or a murine double minute 2 (MDM2) inhibitor). The Bcl-2 inhibitor can be venetoclax, navitoclax, obatoclax, ABT-737, S55746, or sabutoclax. The IAP inhibitor can be AT-406, GDC-0917, LCL-161, GDC-0152, Birinapant, HGS1029, TWX024, or AEG35156. The MDM2 inhibitor can be Nutlin, ATSP-7041, or another apoptosis sensitizer, such as a smac mimetic, a MCL-1 inhibitor, or a Bclxl inhibitor.

This document also features a composition that includes a nucleic acid encoding a chimeric antigen receptor described herein (e.g., a chimeric antigen receptor having the ability to bind to a human TRAILshort polypeptide). The composition further can include a nucleic acid encoding a chimeric antigen receptor comprising an antigen binding domain, a hinge, a transmembrane domain, and one or more signaling domains, wherein the antigen binding domain has the ability to bind to any one of a human CD19 polypeptide, a human BCMA polypeptide, a human TSHR polypeptide, a human EPHA3 polypeptide, a human FGFR2 polypeptide, a human HER2 polypeptide, a human TROP2 polypeptide, a human NY-ESO polypeptide, a human Mesothelin polypeptide, a human EGFR polypeptide, a human EGFRviii polypeptide, a human IL13Ra2 polypeptide, a human Folate receptor alpha polypeptide, a human Folate receptor Beta polypeptide, a human gut integrin (e.g., ITGB7) polypeptide, a human CD103 polypeptide, a human CD83 polypeptide, a human CD22 polypeptide, a human CD20 polypeptide, a human CD79b polypeptide, a human CD79a polypeptide, a human CD123 polypeptide, a human CD33 polypeptide, a human ILR1a polypeptide, a human CD34 polypeptide, a human CD30 polypeptide, a human CD4 polypeptide, a human CD8 polypeptide, a human T cell receptor alpha polypeptide, a human T cell receptor beta polypeptide, a human CD3 polypeptide, a human CD5 polypeptide, a human CD7 polypeptide, a human gp120 polypeptide, a human galactomannan polypeptide, a human PSMA polypeptide, a human MUC polypeptide, a human PD-1 polypeptide, a human CD80 polypeptide, a human CD86 polypeptide, a human CEA polypeptide, a human GPC3 polypeptide, a human ROR1 polypeptide, a human AFP polypeptide, a human CD138 polypeptide, a human CD38 polypeptide, a human CD44v6 polypeptide, a human CD70 polypeptide, a human CLEC12A (CLL-1) polypeptide, a human CS-1 polypeptide, a human FAP polypeptide, a human GPRC5D polypeptide, a human MUC-1 polypeptide, a human MUC16 polypeptide, or a human NKG2D polypeptide. In some cases, the antigen binding domain has the ability to bind to a human CD19 polypeptide.

For example, the composition further can include a nucleic acid encoding a chimeric antigen receptor comprising an antigen binding domain, a hinge, a transmembrane domain, and one or more signaling domains, wherein the antigen binding domain contains the CDRs of a FMC63 scFv antibody and binds to a CD19 antigen, contains the CDRs of a MOR208 scFv and binds to a CD19 antigen, contains the CDRs of a humanized scFv antibody and binds to a CD19 antigen, contains the CDRs of a 4G7 scFv antibody and binds to a CD19 antigen, contains the CDRs of a low affinity scFv antibody and binds to a CD19 antigen, contains the CDRs of a 5E5 scFv antibody and binds to a MUC-1 antigen, contains the CDRs of a 4D5 scFv antibody and binds to a HER-2 antigen, contains the CDRs of a FRP5 scFv antibody and binds to a HER-2 antigen, contains the CDRs of a M27 scFv antibody and binds to a EGFR antigen, contains the CDRs of a Cetuximab scFv antibody and binds to a EGFR antigen, contains the CDRs of a C4 based scFv antibody and binds to a Folate receptor alpha antigen, contains the CDRs of a MOv19 scFv antibody and binds to a Folate receptor alpha antigen, contains the CDRs of a SS1 scFv antibody and binds to a Mesothelin antigen, contains the CDRs of a M clone scFv antibody and binds to a Mesothelin antigen, contains the CDRs of a Amatuximab scFv antibody and binds to a Mesothelin antigen, contains the CDRs of a Anetumab scFv antibody and binds to a Mesothelin antigen, contains the CDRs of a ET1402L1 scFv antibody and binds to a AFP antigen, contains the CDRs of an Anti-CEA scFv antibody and binds to a CEA antigen, contains the CDRs of a CEACAM5 scFv antibody and binds to a CEA antigen, contains the CDRs of a hMN14 scFv antibody and binds to a CEA antigen, contains the CDRs of a 22172,22176 scFv antibody and binds to a CD123 antigen, contains the CDRs of a humanized scFv antibody and binds to a CD123 antigen, contains the CDRs of a humanized scFv antibody and binds to a CD123 antigen, contains the CDRs of a Tagrazofusp based scFv antibody and binds to a CD123 antigen, contains the CDRs of a MY96 scFv antibody and binds to a CD33 antigen, contains the CDRs of a humanized scFv antibody and binds to a CD33 antigen, contains the CDRs of a humanized scFv antibody and binds to a CLEC12A antigen, contains the CDRs of a CLL1 scFv antibody and binds to a CLEC12A antigen, contains the CDRs of a M6E7 scFv antibody and binds to a CLEC12A antigen, contains the CDRs of a m21C9 scFv antibody and binds to a CLEC12A antigen, contains the CDRs of a M20B1 scFv antibody and binds to a CLEC12A antigen, contains the CDRs of a M28H12 scFv antibody and binds to a CLEC12A antigen, contains the CDRs of a M0971 scFv antibody and binds to a CD22 antigen, contains the CDRs of a humanized scFv clone antibody and binds to a CD22 antigen, contains the CDRs of a Inotuzumab based scFv antibody and binds to a CD22 antigen, contains the CDRs of a Moxetumomab based scFv antibody and binds to a CD22 antigen, contains the CDRs of a Rituximab scFv antibody and binds to a CD20 antigen, contains the CDRs of a Leu 16 scFv antibody and binds to a CD20 antigen, contains the CDRs of a CD20 scFv antibody and binds to a CD20 antigen, contains the CDRs of a BCMA-02 scFv antibody and binds to a BCMA antigen, contains the CDRs of a LCAR38 scFv antibody and binds to a BCMA antigen, contains the CDRs of a BCMA scFv antibody and binds to a BCMA antigen, contains the CDRs of a biepitopic scFv antibody and binds to a BCMA antigen, contains the CDRs of a NVS BCMA scFv antibody and binds to a BCMA antigen, contains the CDRs of a CSIR scFv antibody and binds to a CS-1 antigen, contains the CDRs of a CS1 scFv antibody and binds to a CS-1 antigen, contains the CDRs of an Elotuzumab based scFv antibody and binds to a CS-1 antigen, contains the CDRs of a scFv antibody and binds to a CD138 antigen, contains the CDRs of a humanized scFv antibody and binds to a CD44v6 antigen, contains the CDRs of a cMAb U36 scFv antibody and binds to a CD44v6 antigen, contains the CDRs of a scFv antibody and binds to a NKG2D antigen, contains the CDRs of a NKG2Dg scFv antibody and binds to a NKG2D antigen, contains the CDRs of a nanobody CD38cFv antibody and binds to a CD38 antigen, contains the CDRs of a humanized scFv antibody and binds to a CD38 antigen, contains the CDRs of a humanized scFv antibody and binds to a GPRC5D antigen, contains the CDRs of a scFv (L-H) and (H-L) antibody and binds to a CD79b antigen, contains the CDRs of a M290 scFv antibody and binds to a CD103 antigen, contains the CDRs of a FAP5 scFv antibody and binds to a FAP antigen, contains the CDRs of a human CD70 scFv antibody and binds to a CD70 antigen, contains the CDRs of a 4H11 scFv antibody and binds to a MUC16 antigen, contains the CDRs of a IL13R scFv antibody and binds to a IL13Ra2 antigen, contains the CDRs of a Muromonab scFv antibody and binds to a CD3 antigen, contains the CDRs of a Teplizumab scFv antibody and binds to a CD3 antigen, contains the CDRs of a Blinatumomab scFv antibody and binds to a CD3 antigen, contains the CDRs of a Brentuximab scFv antibody and binds to a CD30 antigen, contains the CDRs of a 4C8 scFv antibody and binds to a CD34 antigen, contains the CDRs of an Ibalizumab scFv antibody and binds to a CD4 antigen, contains the CDRs of an Anti-CD5 scFv antibody and binds to a CD5 antigen, contains the CDRs of a 9F2A11 scFv antibody and binds to a CD5 antigen, contains the CDRs of an Ab5D7v scFv antibody and binds to a CD5 antigen, contains the CDRs of a Polatuzumab scFv antibody and binds to a CD7 antigen, contains the CDRs of an Ab 4450 scFv antibody and binds to a CD79a antigen, contains the CDRs of an Anti-CD79a scFv antibody and binds to a CD79a antigen, contains the CDRs of a Crefmirlimab scFv antibody and binds to a CD8 antigen, contains the CDRs of a Galiximab scFv antibody and binds to a CD80 antigen, contains the CDRs of a 3C12 scFv antibody and binds to a CD83 antigen, contains the CDRs of a 32A scFv antibody and binds to a CD86 antigen, contains the CDRs of an Anti-EGFRvIII scFv antibody and binds to an EGFRviii antigen, contains the CDRs of an Ifabotuzumab scFv antibody and binds to an EPHA3 antigen, contains the CDRs of an Aprutumab scFv antibody and binds to a FGFR2 antigen, contains the CDRs of a Bemarituzumab scFv antibody and binds to a FGFR2 antigen, contains the CDRs of a M909 scFv antibody and binds to a Folate receptor Beta antigen, contains the CDRs of an ASO4498 scFv antibody and binds to a Folate receptor Beta antigen, contains the CDRs of an Antibody #2 scFv antibody and binds to a Folate receptor Beta antigen, contains the CDRs of an Antibody #3 scFv antibody and binds to a Folate receptor Beta antigen, contains the CDRs of an EH7 scFv antibody and binds to a galactomannan antigen, contains the CDRs of a BB10 scFv antibody and binds to a galactomannan antigen, contains the CDRs of an Elipovimab scFv antibody and binds to a gp 120 antigen, contains the CDRs of a Suvizumab scFv antibody and binds to a gp 120 antigen, contains the CDRs of a Teropavimab scFv antibody and binds to a gp 120 antigen, contains the CDRs of a Codrituzumab scFv antibody and binds to a GPC3 antigen, contains the CDRs of an Etrolizumab scFv antibody and binds to a gut integrins antigen, contains the CDRs of a 10E12 scFv antibody and binds to an ILR1a antigen, contains the CDRs of a 9E11 scFv antibody and binds to an ILR1a antigen, contains the CDRs of a 9G5 scFv antibody and binds to an ILR1a antigen, contains the CDRs of a Cantuzumab scFv antibody and binds to a MUC antigen, contains the CDRs of a Clivatuzumab scFv antibody and binds to a MUC antigen, contains the CDRs of a Gatipotuzumab scFv antibody and binds to a MUC antigen, contains the CDRs of a Sofituzumab scFv antibody and binds to a MUC antigen, contains the CDRs of a Ubamatamab scFv antibody and binds to a MUC antigen, contains the CDRs of a 12D7 scFv antibody and binds to a NY-ESO antigen, contains the CDRs of a T1 scFv antibody and binds to a NY-ESO antigen, contains the CDRs of a T2 scFv antibody and binds to a NY-ESO antigen, contains the CDRs of a T3 scFv antibody and binds to a NY-ESO antigen, contains the CDRs of a Nivolumab scFv antibody and binds to a PD-1 antigen, contains the CDRs of a Pembrolizumab scFv antibody and binds to a PD-1 antigen, contains the CDRs of a J591 scFv antibody and binds to a PSMA antigen, contains the CDRs of a Zilovertamab scFv antibody and binds to a ROR1 antigen, contains the CDRs of an Anti-TCRa scFv antibody and binds to a T cell receptor alpha antigen, contains the CDRs of an Anti-TCRBC1 scFv antibody and binds to a T cell receptor beta antigen, contains the CDRs of a K1-18 scFv antibody and binds to a TSHR antigen, contains the CDRs of a Sacituzumab scFv antibody and binds to a TROP2 antigen, or contains the CDRs of a Datopotamab scFv antibody and binds to a TROP2 antigen. In some cases, the composition further includes a nucleic acid encoding a chimeric antigen receptor comprising an antigen binding domain, a hinge, a transmembrane domain, and one or more signaling domains, wherein the antigen binding domain includes the CDRs of a FMC63 scFv antibody and binds to a CD19 antigen, comprises the CDRs of a MOR208 scFv and binds to a CD19 antigen, includes the CDRs of a humanized scFv antibody and binds to a CD19 antigen, includes the CDRs of a 4G7 scFv antibody and binds to a CD19 antigen, or includes the CDRs of a low affinity scFv antibody and binds to a CD19 antigen.

A method of treating a mammal (e.g., a human) having cancer also is featured. The method includes administering, to the mammal (e.g., a human), a composition described herein (e.g., a composition that includes a population of cells described herein or a composition that includes a nucleic acid encoding a chimeric antigen receptor described herein). The cancer can be a TRAILshort+ cancer such as TRAILshort squamous cell carcinoma, lymphoma, cervical cancer, renal cell carcinoma, breast cancer, prostate cancer, ovarian cancer, lung cancer, bladder cancer, head and neck cancer, uterine cancer, esophageal cancer, stomach cancer, colorectal cancer, sarcoma, or pancreatic cancer. The number of cancer cells within the mammal (e.g., human) can be reduced following the administering step. The method can include administering, to the mammal, a pro-apoptotic compound (e.g., a Bcl-2 inhibitor, an IAP inhibitor, a MDM2 inhibitor, a smac mimetic, a Bcxlx inhibitor, or a MCL-1 inhibitor).

In another aspect, a method of treating a mammal (e.g., human) having an infection is featured. The method includes administering, to the mammal (e.g., human), a composition described herein (e.g., a composition that includes a population of cells described herein or a composition that includes a nucleic acid encoding a chimeric antigen receptor described herein). The infection can be a chronic infection. The infection can be selected from the group consisting of human immunodeficiency virus (HIV) infection, hepatitis B virus (HBV) infection, hepatitis C virus (HCV) infection, human papilloma virus (HPV) infection, tuberculosis (TB) infection, cytomegalovirus (CMV) infection, and Epstein-Barr virus (EBV) infection.

This document also features a method for binding a chimeric antigen receptor to a TRAILshort polypeptide. The method includes contacting the TRAILshort polypeptide with a chimeric antigen receptor described herein. The contacting can be performed in vitro or in vivo. For example, the contacting can be performed within a mammal (e.g., human) by administering a cell comprising the chimeric antigen receptor to the mammal (e.g., human).

Unless otherwise defined, all technical and scientific terms used herein have the same meaning as commonly understood by one of ordinary skill in the art to which this disclosure pertains. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows an amino acid sequence of a TRAILshort polypeptide (101 amino acids, SEQ ID NO:34). The first 90 amino acids are the same as a full-length TRAIL polypeptide, while the last 11 amino acids of the C-terminus are distinct from a full-length TRAIL polypeptide (SEQ ID NO:35).

FIG. 2 contains an amino acid sequence of a scFv of FMC63 (SEQ ID NO:36) and a nucleic acid sequence encoding FMC63 (SEQ ID NO:37).

FIG. 3A contains amino acid sequences of murine and humanized anti-TRAILshort heavy chains, including amino acid sequences of murine heavy chain HC0 (SEQ ID NO:17) and humanized heavy chains HC1 (SEQ ID NO:19), HC2 (SEQ ID NO: 21), HC3 (SEQ ID NO:22), and HC4 (SEQ ID NO:24). FIG. 3A also contains amino acid sequences of the murine and humanized light chains, including the amino acid sequence of murine light chain LC0 (SEQ ID NO:26), LC1 (SEQ ID NO:28), LC2 (SEQ ID NO: 29), LC3 (SEQ ID NO:31), and LC4 (SEQ ID NO:33).

FIG. 3B contains the CDRs and framework region amino acid sequences of HC3 and LC2.

FIG. 3C contains an amino acid sequence of the variable region of murine TRAILs 2.2 VH0 (SEQ ID NO:16) and an amino acid sequence of the variable region of the humanized variants VH1 (SEQ ID NO:18), VH2 (SEQ ID NO:20), VH3 (SEQ ID NO: 8), and VH4 (SEQ ID NO:23). FIG. 3C also contains an amino acid sequence of the variable region of murine TRAILs 2.2 VL0 (SEQ ID NO:25) and an amino acid sequence of the variable region of the humanized variants VL1 (SEQ ID NO:27), VL2 (SEQ ID NO: 15), VL3 (SEQ ID NO:30), and VL4 (SEQ ID NO:32). The CDRs based on the Kabat numbering system are indicated in bold type and the CDRs from the IMGT numbering system are underlined.

FIG. 4 contains nucleic acid sequences encoding a ScFv portion of the pLV HC3-LC2-K161 TRAILshort CAR, including a nucleic acid sequence encoding HC3 (SEQ ID NO: 38) and a codon optimized (SEQ ID NO:39) sequence encoding HC3, and the nucleic acid sequence encoding LC2 (SEQ ID NO:40) and a codon optimized (SEQ ID NO:41) sequence encoding LC2.

FIG. 5 contains a nucleic acid sequence encoding a ScFv portion of pLV LC2-HC2 K163 TRAILshort CAR, including a nucleic acid sequence encoding HC2 (SEQ ID NO: 42) and a codon optimized (SEQ ID NO:43) sequence encoding HC2, and a nucleic acid sequence encoding LC2 (SEQ ID NO:40) and a codon optimized (SEQ ID NO:41) sequence encoding LC2.

FIG. 6 contains a nucleic acid sequence (SEQ ID NO:44) encoding a CD8 leader, a nucleic acid sequence (SEQ ID NO:46) encoding a linker, a nucleic acid sequence (SEQ ID NO:48) encoding a CD8 hinge region, a nucleic acid sequence (SEQ ID NO:50) encoding a CD8 transmembrane (TM) domain, a nucleic acid sequence (SEQ ID NO:52) encoding a 4-1BB intracellular signaling domain, a nucleic acid sequence (SEQ ID NO: 54) encoding a CD28 hinge region, a nucleic acid sequence (SEQ ID NO:56) encoding a CD28 TM domain, a nucleic acid sequence (SEQ ID NO:58) encoding a CD28 intracellular signaling domain, and a nucleic acid sequence (SEQ ID NO:60) encoding a CD3ζ intracellular signaling domain.

FIG. 7A contains an amino acid sequence of each of the following: a CD8 leader (SEQ ID NO:45), a linker (SEQ ID NO:47), a CD8 hinge region (SEQ ID NO:49), a CD8 TM domain (SEQ ID NO:51), a 4-1BB intracellular signaling domain (SEQ ID NO:53), a CD28 hinge region (SEQ ID NO:55), a CD28 TM domain (SEQ ID NO:57), a CD28 intracellular signaling domain (SEQ ID NO:59), and a CD3ζ intracellular signaling domain (SEQ ID NO:61). FIG. 7A also contains additional exemplary hinges for CARs (in some cases, these hinges can be used as linkers) (SEQ ID NOs: 62-69).

FIG. 7B contains a schematic of a TRAILshort (TS) CAR and a CD19 CAR.

FIG. 7C contains exemplary transmembrane domains for CARs (SEQ ID NOs: 70-75).

FIG. 7D contains exemplary intracellular signaling domains for CARS (SEQ ID NOs: 76-84).

FIG. 8 contains graphs showing TsCAR T cells did not target total T cells (upper left), CD4+ T cells (upper right) or CD8+ T cells (bottom left). The graphs are presented as the percent of basal target T cells at different ratios of the CAR T cells (HC3-LC2-K161) or untransduced (UTD) cells to target cells. The number of the target T cells cultured alone was considered as 100% and the number of total T cells, CD4⁺ T cells, and CD8⁺ T cells were calculated as a percentage of the T cells cultured alone. The measurements were taken at 24 hours. Similar results were found at both 48 and 72 hours.

FIG. 9 contains graphs showing TsCAR T cell proliferation upon incubation with T cells was similar to untransduced (UTD) cell proliferation, indicating a lack of antigen specific activation of TsCAR T cells after 24 hours. The upper left panel shows the absolute number of CAR T cells, the upper right panel shows the absolute number of target cells, the lower left panel shows the absolute number of target CD4⁻ cells, and the lower right panel shows the absolute number of target CD8⁺ cells.

FIG. 10 contains graphs showing that TsCAR T cells exhibited potent antitumor efficacy against ARG77 U (plasma cell leukemia; upper left), BCWM (Waldenstrom macroglobulinemia; upper right), Je-Kol (lymphoma; lower left), and Jurkat (acute T cell leukemia; lower right) cell lines. T cell activation (PMA pre-treatment) increased TRAILshort expression and increased TsCAR T cell cytotoxicity (bottom right).

FIG. 11 contains graphs showing TsCAR T cells were effective against WT Hela cells, especially at higher E:T ratios (upper panels). However TsCAR did not have cytotoxic activity against TRAIL knockout (TKO) cell lines (bottom panels), demonstrating the specificity of TsCAR T cells.

FIG. 12 contains graphs showing that T cells expressing both CAR19 and TsCAR (Dual CART cells) were cytotoxic to the cancer cell lines ARH77 and JeKo-1 in vitro. UTD refers to untransduced cells. The top graph shows data from ARH77 and BCWM cells. The middle graph shows data from HeLa and HeLa TKO cells. The bottom graph shows data from JeKo-1 cells.

FIG. 13 contains graphs showing that Dual CART cells were activated when co-cultured with ARH77, BCWM, and JeKo-1 cell lines.

FIG. 14 contains graphs showing the tumor load was decreased after transplanting dual CART cells in a lymphoma transplantation model. 1M JeKo-1 Luc and 1M UTD or CART cells were transplanted to NOD scid gamma (NSG™) immunodeficient mice (The Jackson Laboratory; Bar Harbor, ME). The dual CART cells decreased the tumor burden in mice as early as day 15 compared to the other CART cell groups (top graphs; * p<0.05, ** p<0.01, *** p<0.001, one-way ANOVA; error bars, SEM, n=5 mice). Thus, the dual CAR T cells exhibited better anti-tumor activity as compared to either CAR T cells alone in vivo in a lymphoma transplantation model. Mice were bled at day 22 to count the circulating CART cells in each group. The CART19 and dual CART cell group had detectable levels of CART cells (bottom left). FIG. 14 also contains survival curves after transplantation with untransduced cells (UTD), TsCAR T cells (CARTS1), CD19 CAR T cells (CAR19), or dual CAR T cells that expressed both the TS and CD19 CARs. The Dual CART cell group had a higher probability of survival than the UTD group (bottom right; ** p<0.01, Log-rank (Mantel-Cox) test; error bars, SEM).

FIG. 15 contains a graph demonstrating that TsCAR T cells combined with Navitoclax (BCL-2 inhibitor) treatment resulted in decreased CAR T cell proliferation against JeKo-1 cell line. The combination of the TsCAR T cells with Venetoclax (BCL-2 inhibitor) did not lead to decreased proliferation.

FIG. 16 contains graphs showing that a combination of BCL-2 inhibitors (venetoclax and navitoclax) and TsCAR T cells resulted in increased CAR T cell cytotoxicity against the JeKo-1 cell line. The upper horizontal line indicates to Venetoclax killing JeKo-1 cells when cultured together and the lower horizontal line indicates to Navitoclax killing JeKo-1 cells when cultured together.

FIGS. 17A-17F show the dose dependent efficacy of TsCAR T cells (CARTS1) in a mouse xenograft model. FIG. 17A is a schematic summarizing the in vivo experiments. 1M JeKo-1 Luciferase cells were transplanted to NSG mice at day-14. The mice were randomized at day-1, and either 1M (n=10 mice) or 5M (n=5 mice) UTD or CARTS1 cell were transplanted at day 0. FIG. 17B is a graph plotting bioluminescence after luciferin injection to the mice that were transplanted with either 1M UTD or CARTS1 cells, plotted according to the time (n=10 mice/group). FIG. 17C is a graph plotting survival, showing that all of the mice died between days 18 and 25. FIG. 17D is a graph plotting bioluminescence of the UTD and CARTS1 mice that were transplanted with either 5M of UTD or CARTS1 cell. CARTS1 mice had significantly less tumor burden starting on day 16 (** p<0.01, **** p<0.0001, Student's t test; error bars, SEM; n=5 mice). FIG. 17E is a graph plotting survival, showing that the CARTS1 (5M) mice showed increased survival compared to the UTD (5M) mice (** p<0.01, Log-rank (Mantel-Cox) test; error bars, SEM). The CARTS1 mice still had low tumor burden when they were sacrificed at day 55 due to graft versus host disease (GVHD). GVHD was an expected end point, as human T cell derived CART cells were transplanted into the mice. FIG. 17F is a graph plotting CART cell numbers in peripheral blood samples collected from the mice at day 21 via tail vein bleeding. The amount of CART cell in each mouse was plotted. The CARTS1 (5M) mice had significantly higher number of CART cells than the UTD controls (*p<0.05 Student's t test; error bars, SEM; n=5 mice). No T cells were detected in UTD (1M), CARTS (1M), or UTD (5M) mice that survived up to 21 days (not shown).

### DETAILED DESCRIPTION

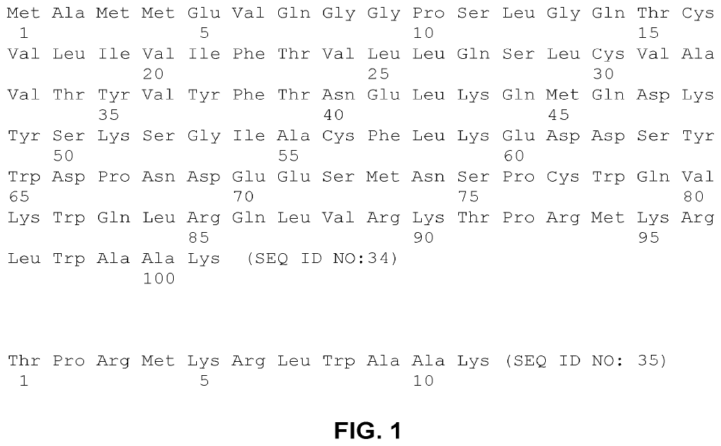

This document provides chimeric antigen receptors (CARs) that bind (e.g., specifically bind) to a TRAILshort polypeptide (e.g., a human TRAILshort polypeptide). For example, the document provides CARs that bind (e.g., specifically bind) to a polypeptide comprising, consisting essentially of, or consisting of the TRAILshort amino acid set forth in FIG. 1 (e.g., SEQ ID NO:34) or the 11 C-terminal amino acids unique to TRAILshort (SEQ ID NO:35, FIG. 1). The generation of TRAILshort antibodies that are specific to the 11 C terminal amino acids unique to TRAILshort is described, for example, in Schnepple et al., 2011 J Biol Chem 286:35742-35754, and U.S. Pat. No. 11,136,402. Targeting a TRAILshort polypeptide with the CARs described herein can be used to, for example, increase apoptosis of TRAILshort+ cells (e.g., cancer cells or infected cells).

The term “antibody” as used herein includes polyclonal antibodies, monoclonal antibodies, recombinant antibodies, humanized antibodies, human antibodies, chimeric antibodies, multi-specific antibodies (e.g., bispecific antibodies) formed from at least two antibodies, diabodies, single-chain variable fragment antibodies (e.g., scFv antibodies), and tandem single-chain variable fragments antibody (e.g., taFv). A diabody can include two chains, each having a heavy chain variable domain and a light chain variable domain, either from the same or from different antibodies (see, e.g., Hornig and Färber-Schwarz, Methods Mol. Biol., 907:713-27 (2012); and Brinkmann and Kontermann, MAbs., 9(2): 182-212 (2017)). The two variable regions can be connected by a polypeptide linker (e.g., a polypeptide linker having five to ten residues in length). In some cases, an interdomain disulfide bond can be present in one or both of the heavy chain variable domain and light chain variable domain pairs of the diabody. A scFv is a single-chain polypeptide antibody in which the heavy chain variable domain and the light chain variable domain are directly connected or connected via a polypeptide linker (e.g., a polypeptide linker having eight to 18 residues in length). See, also, Chen et al., Adv. Drug Deliv. Rev., 65(10): 1357-1369 (2013). A scFv can be designed to have an orientation with the heavy chain variable domain being followed by the light chain variable domain or can be designed to have an orientation with the light chain variable domain being followed by the heavy chain variable domain. In both cases, the optional linker can be located between the two domains.

An antibody provided herein can include the CDRs as described herein (e.g., as described in Table 1) and can be configured to be a murine antibody, a humanized antibody, or a chimeric antibody. In some cases, an antibody provided herein can include the CDRs as described herein (e.g., as described in Table 1) and can be a monoclonal antibody. In some cases, an antibody provided herein can include the CDRs as described herein (e.g., as described in Table 1) and can be configured as a scFv antibody.

The term “antigen binding fragment” as used herein refers to a fragment of an antibody (e.g., a fragment of a humanized antibody, a fragment of a murine antibody, or a fragment of a chimeric antibody) having the ability to bind to an antigen. Examples of antigen binding fragments include, without limitation, Fab, Fab′, or F(ab′)₂ antigen binding fragments. An antigen binding fragment provided herein can include the CDRs as described herein (e.g., as described in Table 1) and can be configured to be a murine antigen binding fragment, a humanized antigen binding fragment, or a chimeric antigen binding fragment. In some cases, an antigen binding fragment provided herein can include the CDRs as described herein (e.g., as described in Table 1) and can be a monoclonal antigen binding fragment. In some cases, an antigen binding fragment provided herein can include the CDRs as described herein (e.g., as described in Table 1) and can be configured as a Fab antibody. In some cases, a Fab antibody can include a partial hinge sequence for disulfide bonding between heavy and light chains of the Fab.

The term “antibody domain” as used herein refers to a domain of an antibody such as a heavy chain variable domain (VH domain) or a light chain variable domain (VL domain) in the absence of one or more other domains of an antibody. In some cases, an antibody domain can be a single antibody domain (e.g., a VH domain or a VL domain) having the ability to bind to an antigen. An antibody domain provided herein can include the CDRs as described herein (e.g., as described in Table 1) and can be a murine antibody domain, a human VH domain), a humanized antibody domain (e.g., a humanized VH domain), or a chimeric antibody domain (e.g., a chimeric VH domain). In some cases, an antibody domain provided herein can include the CDRs as described herein (e.g., as described in Table 1) and can be a monoclonal antibody domain. In some cases, an antibody domain provided herein can include the CDRs as described herein (e.g., as described in Table 1) and can be engineered as a single VH domain or a single VL domain.

An anti-TRAILshort antibody, anti-TRAILshort antigen binding fragment, or anti-TRAILshort antibody domain provided herein can be of the IgA-, IgD-, IgE-, IgG-, or IgM-type, including IgG- or IgM-types such as, without limitation, IgG1-, IgG2-, IgG3-, IgG4-, IgM1-, and IgM2-types. In some cases, an antibody provided herein (e.g., an anti-TRAILshort antibody) can be an scFv antibody. In some cases, an antigen binding fragment provided herein (e.g., an anti-TRAILshort antibody fragment) can be a Fab. In some cases, an antibody provided herein (e.g., an anti-TRAILshort antibody) can be a fully intact antibody. In some cases, an antibody domain provided herein (e.g., an anti-TRAILshort antibody domain) can be a VH domain.

The term “chimeric antigen receptor” as used herein refers to a chimeric polypeptide that is designed to include an optional signal peptide, an antigen binding domain, an optional hinge, a transmembrane domain, and one or more intracellular signaling domains. As described herein, the antigen binding domain of a CAR provided herein can be designed to bind to a TRAILshort polypeptide (e.g., a human TRAILshort polypeptide). For example, a CAR provided herein can be designed to include the components of an antibody, antigen binding fragment, and/or antibody domain described herein (e.g., a combination of CDRs) as an antigen binding domain provided that that antigen binding domain has the ability to bind to a TRAILshort polypeptide (e.g., a human TRAILshort polypeptide). In some examples, a CAR provided herein can be designed to include an antigen binding domain that includes two sets of three CDRs (e.g., CDR1, CDR2, and CDR3 of a heavy chain and CDR1, CDR2, and CDR3 of a light chain) of an antigen binding fragment provided herein (e.g., SEQ ID NOs: 1-3 and SEQ ID NO: 9, the amino acid sequence GAS, and SEQ ID NO:10). In some cases, an antigen binding domain of a CAR targeting a TRAILshort polypeptide can be designed to include a VH domain described herein or a scFv antibody described herein.

In some cases, a CAR provided herein can be designed to include a signal peptide. Any appropriate signal peptide can be used to design a CAR described herein. Examples of signal peptide that can be used to make a CAR described herein include, without limitation, a tPA signal peptide, BiP signal peptide, or CD8a signal peptide.

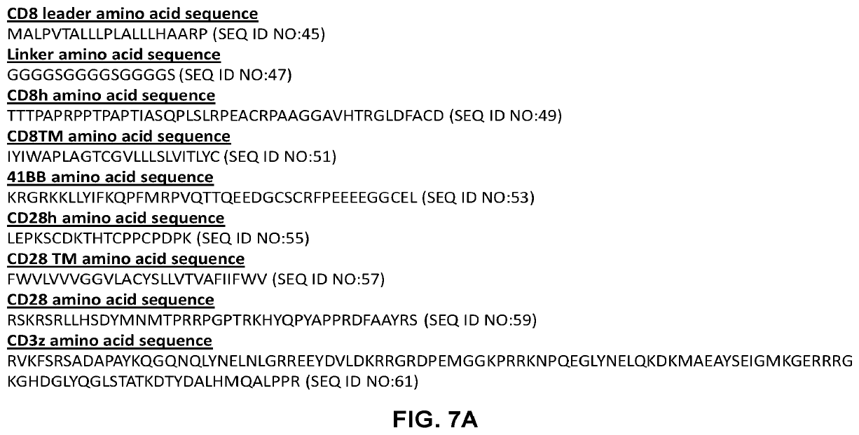

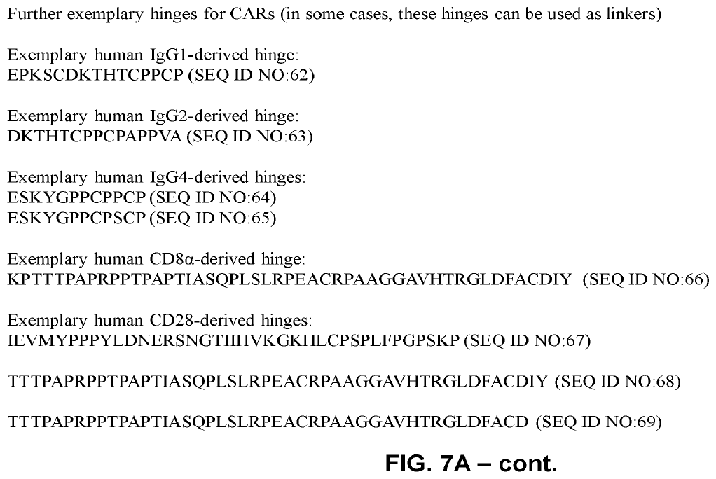

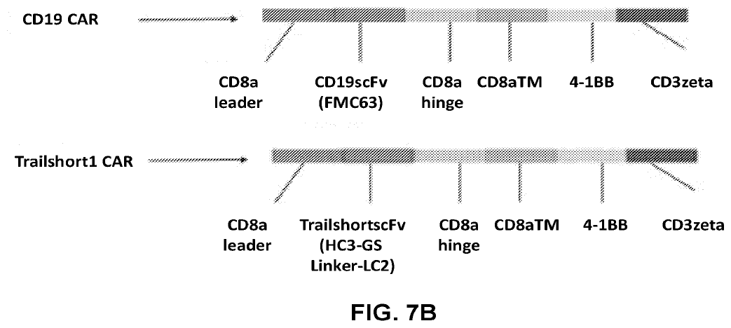

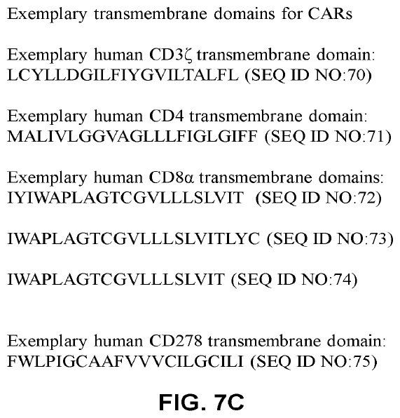

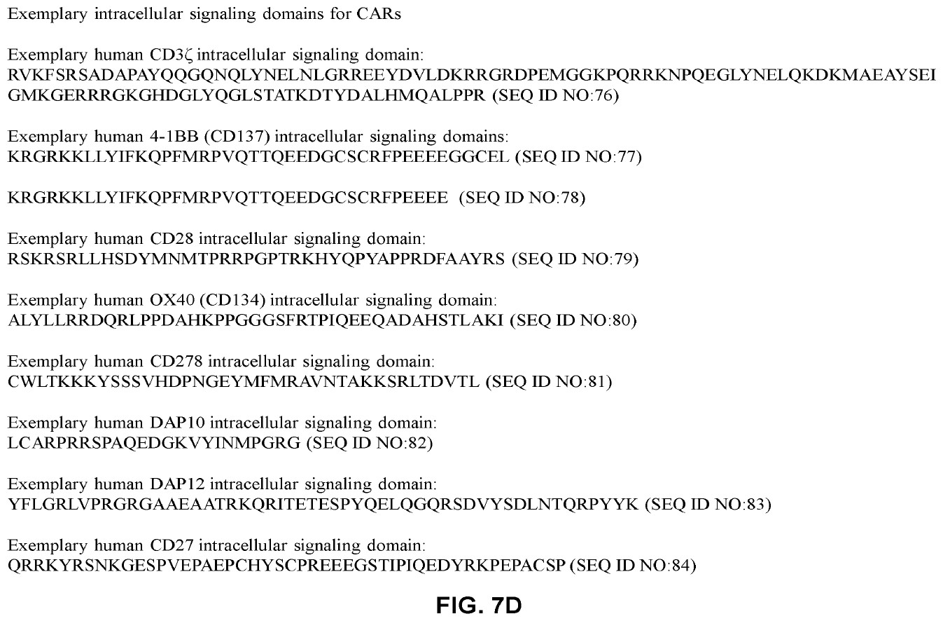

In some cases, a CAR provided herein can be designed to include a hinge. Any appropriate hinge can be used to design a CAR described herein. Examples of hinges that can be used to make a CAR described herein include, without limitation, Ig-derived hinges (e.g., an IgG1-derived hinge, an IgG2-derived hinge, or an IgG4-derived hinge), Ig-derived hinges containing a CD2 domain and a CD3 domain, Ig-derived hinges containing a CD2 domain and lacking a CD3 domain, Ig-derived hinges containing a CD3 domain and lacking a CD2 domain, Ig-derived hinges lacking a CD2 domain and lacking a CD3 domain, CD8α-derived hinges, CD28-derived hinges, and CD3ζ-derived hinges. See, e.g., the exemplary hinges of FIG. 7A. A CAR provided herein can be designed to include a hinge of any appropriate length. For example, a CAR provided herein can be designed to include a hinge that is from about 3 to about 75 (e.g., from about 3 to about 65, from about 3 to about 50, from about 5 to about 75, from about 10 to about 75, from about 5 to about 50, from about 10 to about 50, from about 10 to about 40, or from about 10 to about 30) amino acid residues in length. In some cases, a linker sequence (e.g., (GGGGS)₂₋₅; SEQ ID NOS: 157, 47, 158, and 159, respectively) can be used as a hinge to make a CAR described herein.

A CAR provided herein can be designed to include any appropriate transmembrane domain. See, e.g., the exemplary transmembrane domains of FIGS. 7A and 7C. For example, the transmembrane domain of a CAR provided herein can be, without limitation, a CD3ζ transmembrane domain, a CD4 transmembrane domain, a CD8α transmembrane domain, a CD28 transmembrane domain, or a 4-1BB transmembrane domain.

A CAR provided herein can be designed to include one or more intracellular signaling domains. See, e.g., the exemplary intracellular domains of FIGS. 7A and 7D. For example, a CAR provided herein can be designed to include one, two, three, or four intracellular signaling domains. Any appropriate intracellular signaling domain or combination of intracellular signaling domains can be used to make a CAR described herein. Examples of intracellular signaling domains that can be used to make a CAR described herein include, without limitation, CD3ζ intracellular signaling domains, CD27 intracellular signaling domains, CD28 intracellular signaling domains, OX40 (CD134) intracellular signaling domains, 4-1BB (CD137) intracellular signaling domains, CD278 intracellular signaling domains, DAP10 intracellular signaling domains, and DAP12 intracellular signaling domains. In some cases, a CAR described herein can be designed to be a first generation CAR having a CD3ζ intracellular signaling domain. In some cases, a CAR described herein can be designed to be a second generation CAR having a CD28 intracellular signaling domain followed by a CD3ζ intracellular signaling domain. In some cases, a CAR described herein can be designed to be a third generation CAR having (a) a CD28 intracellular signaling domain followed by (b) a CD27 intracellular signaling domain, an OX40 intracellular signaling domains, or a 4-1BB intracellular signaling domain followed by (c) a CD3ζ intracellular signaling domain. See, e.g., Feins et al., Am J Hematol., 94(S1): S3-S9 (2019).

In some cases, a CAR targeting a TRAILshort polypeptide can be designed to include an scFv having a heavy chain variable domain comprising SEQ ID NO:1, SEQ ID NO: 2, and SEQ ID NO:3, followed by a linker such as (GGGGS)₂₋₅ (SEQ ID NOs: 157, 47, 158, and 159, respectively), followed by a light chain variable domain comprising SEQ ID NO:9, the amino acid sequence GAS, and SEQ ID NO:10, followed by a hinge such as a hinge/linker (e.g., an IgG4-derived hinge, a CD8a hinge, or a linker plus IgG4-derived hinge), followed by a transmembrane domain (e.g., a human CD28 transmembrane domain or a CD8a transmembrane domain), followed by one or more intracellular signaling domains.

In some cases, a CDR1 of a heavy chain variable domain of an antibody that can bind to a TRAILshort polypeptide can be GYIFTNND (SEQ ID NO:1). In some cases, a CDR1 of a heavy chain variable domain of an antibody that can bind to a TRAILshort polypeptide can be NNDMN (SEQ ID NO:85). Other examples of a CDR1 of a heavy chain variable domain of an antibody that can bind to a TRAILshort polypeptide include, without limitation, GYIFTNN (SEQ ID NO:86), GYIFTNNDM (SEQ ID NO:87), YIFTNNDMN (SEQ ID NO:88), YIFTNNDM (SEQ ID NO:89), YIFTNND (SEQ ID NO: 90), IFTNNDMN (SEQ ID NO:91), IFTNNDM (SEQ ID NO:92), IFTNND (SEQ ID NO: 93), FTNNDMN (SEQ ID NO:94), FTNNDM (SEQ ID NO:95), FTNND (SEQ ID NO: 96), TNNDMN (SEQ ID NO:97), TNNDM (SEQ ID NO:98), and TNND (SEQ ID NO: 99). In some cases, a CDR2 of a heavy chain variable domain of an antibody that can bind to a TRAILshort polypeptide can be IDPGDGRTK (SEQ ID NO:2). In some cases, a CDR2 of a heavy chain variable domain of an antibody that can bind to a TRAILshort polypeptide can be GIDPGDGRTKYNEKFKG (SEQ ID NO:100). Other examples of a CDR2 of a heavy chain variable domain of an antibody that can bind to a TRAIL short polypeptide include, without limitation, IDPGDGRT (SEQ ID NO:101), IDPGDGRTK (SEQ ID NO:102), IDPGDGRTKYN (SEQ ID NO:103), GIDPGDGRT (SEQ ID NO: 104), IDPGDGR (SEQ ID NO:105), DPGDGRTKYN (SEQ ID NO:106), DPGDGRTKY (SEQ ID NO:107), PGDGRTKYNE (SEQ ID NO: 108), PGDGRTKYN (SEQ ID NO:109), GDGRTKYNEKFKG (SEQ ID NO:110), GDGRTKYNEKFK (SEQ ID NO: 111), IDPGDGRTKYNEKFK (SEQ ID NO:112), DPGDGRTKYNEKF (SEQ ID NO: 113), and PGDGRTKYNEK (SEQ ID NO:114). In some cases, a CDR3 of a heavy chain variable domain of an antibody that can bind to a TRAILshort polypeptide can be GRGGYEFGIDY (SEQ ID NO:3). In some cases, a CDR3 of a heavy chain variable domain of an antibody that can bind to a TRAILshort polypeptide can be GGYEFGIDY (SEQ ID NO:115). Other examples of a CDR3 of a heavy chain variable domain of an antibody that can bind to a TRAILshort polypeptide include, without limitation, GRGGYEFGID (SEQ ID NO:116), GRGGYEFGI (SEQ ID NO:117), GRGGYEFG (SEQ ID NO:118), GRGGYEF (SEQ ID NO:119), RGGYEFGIDY (SEQ ID NO:120), RGGYEFGI (SEQ ID NO:121), RGGYEFGID (SEQ ID NO:122), GGYEFGID (SEQ ID NO: 123), GGYEFGI (SEQ ID NO:124), GYEFGIDY (SEQ ID NO:125), GYEFGID (SEQ ID NO:126), YEFGIDY (SEQ ID NO:127), and YEFGID (SEQ ID NO:128).

In some cases, a CDR1 of a light chain variable domain of an antibody that can bind to a TRAILshort polypeptide can be QSLLNSGNQKNS (SEQ ID NO:9). In some cases, a CDR1 of a light chain variable domain of an antibody that can bind to a TRAILshort polypeptide can be KSSQSLLNSGNQKNSLA (SEQ ID NO:129). Other examples of a CDR1 of a light chain variable domain of an antibody that can bind to a TRAILshort polypeptide include, without limitation, QSLLNSGNQKNSL (SEQ ID NO: 130), QSLLNSGNQKNSLA (SEQ ID NO:131), SQSLLNSGNQKNS (SEQ ID NO: 132), SQSLLNSGNQKNSL (SEQ ID NO:133), SQSLLNSGNQKNSLA (SEQ ID NO: 134), SSQSLLNSGNQKNS (SEQ ID NO:135), SSQSLLNSGNQKNSL (SEQ ID NO: 136), SSQSLLNSGNQKNSLA (SEQ ID NO:137), KSSQSLLNSGNQKNS (SEQ ID NO: 138), KSSQSLLNSGNQKNSL (SEQ ID NO:139), SLLNSGNQKNSLA (SEQ ID NO: 140), SLLNSGNQKNSL (SEQ ID NO:141), and SLLNSGNQKNS (SEQ ID NO: 142).

In some cases, a CDR2 of a light chain variable domain of an antibody that can bind to a TRAILshort polypeptide can be GAS. In some cases, a CDR2 of a light chain variable domain of an antibody that can bind to a TRAILshort polypeptide can be GASTRES (SEQ ID NO:143). Other examples of a CDR2 of a light chain variable domain of an antibody that can bind to a TRAILshort polypeptide include, without limitation, GAST (SEQ ID NO:144), GASTR (SEQ ID NO:145), GASTRE (SEQ ID NO: 146), ASTRES (SEQ ID NO:147), ASTRE (SEQ ID NO:148), and ASTR (SEQ ID NO: 149).

In some cases, a CDR3 of a light chain variable domain of an antibody that can bind to a TRAILshort polypeptide can be QNDHSFPLT (SEQ ID NO:10). In some cases, a CDR3 of a light chain variable domain of an antibody that can bind to a TRAILshort polypeptide can be QNDHSFPL (SEQ ID NO:150). Other examples of a CDR3 of a light chain variable domain of an antibody that can bind to a TRAILshort polypeptide include, without limitation, QNDHSFP (SEQ ID NO:151), NDHSFPLT (SEQ ID NO:152), NDHSFPL (SEQ ID NO: 153), DHSFPLT (SEQ ID NO:154), and DHSFPL (SEQ ID NO: 155).

In some cases, a CAR targeting a TRAILshort polypeptide can be designed to include an scFv having a heavy chain variable domain comprising SEQ ID NO:8, followed by a linker such as (GGGGS)₂₋₅ (SEQ ID NOS: 157, 47, 158, and 159, respectively), followed by a light chain variable domain comprising SEQ ID NO:15, followed by a hinge such as a hinge/linker (e.g., an IgG4-derived hinge, a CD8a hinge, or a linker plus IgG4-derived hinge), followed by a transmembrane domain, followed by one or more intracellular signaling domains such as one or more intracellular signaling domain.

In some cases, a CAR targeting a TRAILshort polypeptide can be designed to include an scFv having a light chain variable domain comprising SEQ ID NO:9, the amino acid sequence GAS, and SEQ ID NO:10, followed by a linker such as (GGGGS)₂₋₅ (SEQ ID NOS: 157, 47, 158, and 159, respectively), followed by a heavy chain variable domain comprising SEQ ID NO:1, SEQ ID NO:2, and SEQ ID NO:3, followed by a hinge such as a hinge/linker (e.g., an IgG4-derived hinge, a CD8a hinge, or a linker plus IgG4-derived hinge), followed by a transmembrane domain, followed by one or more intracellular signaling domains.

In some cases, a CAR targeting a TRAILshort polypeptide can be designed to include an scFv having a light chain variable domain comprising SEQ ID NO:15, followed by a linker such as (GGGGS)₂₋₅ (SEQ ID NOS: 157, 47, 158, and 159, respectively), followed by a heavy chain variable domain comprising SEQ ID NO:8, followed by a hinge such as a hinge/linker (e.g., an IgG4-derived hinge, a CD8a hinge, or a linker plus IgG4-derived hinge), followed by a transmembrane domain (e.g., a human CD28 transmembrane domain or a CD8a transmembrane domain), followed by one or more intracellular signaling domains.

In one embodiment, a CAR provided herein having the ability to bind to a TRAILshort polypeptide (e.g., a human TRAILshort polypeptide) can include (i) a heavy chain variable domain having a CDR1 having the amino acid sequence set forth in SEQ ID NO: 1 (or a variant of SEQ ID NO:1 with one or two amino acid modifications), a CDR2 having the amino acid sequence set forth in SEQ ID NO:2 (or a variant of SEQ ID NO: 2 with one or two amino acid modifications), and a CDR3 having the amino acid sequence set forth in SEQ ID NO:3 (or a variant of SEQ ID NO:3 with one or two amino acid modifications); and/or (ii) a light chain variable domain having a CDR1 having the amino acid sequence set forth in SEQ ID NO:9 (or a variant of SEQ ID NO:9 with one or two amino acid modifications), a CDR2 having the amino acid sequence GAS (or a variant of GAS with one amino acid modification), and a CDR3 having the amino acid sequence set forth SEQ ID NO:10 (or a variant of SEQ ID NO:10 with one or two amino acid modifications).

In some cases, a CAR provided herein having the ability to bind to a TRAILshort polypeptide (e.g., a human TRAILshort polypeptide) and (a) a heavy chain variable domain having a CDR1 having the amino acid sequence set forth in SEQ ID NO:1 (or a variant of SEQ ID NO:1 with one or two amino acid modifications), a CDR2 having the amino acid sequence set forth in SEQ ID NO:2 (or a variant of SEQ ID NO:2 with one or two amino acid modifications), and a CDR3 having the amino acid sequence set forth in SEQ ID NO:3 (or a variant of SEQ ID NO:3 with one or two amino acid modifications) and/or (b) a light chain variable domain having a CDR1 having the amino acid sequence set forth in SEQ ID NO:9 (or a variant of SEQ ID NO:9 with one or two amino acid modifications), a CDR2 having the amino acid sequence GAS (or a variant of GAS with one amino acid modification), and a CDR3 having the amino acid sequence set forth SEQ ID NO: 10 (or a variant of SEQ ID NO:10 with one or two amino acid modifications) can include any appropriate framework regions. For example, an antigen binding domain of a CAR provided herein can include (a) a heavy chain variable domain that includes a framework region 1 having the amino acid sequence set forth in SEQ ID NO:4 (or a variant of SEQ ID NO:4 with one, two, three, four, five, six, seven, eight, nine, ten, or more amino acid modifications), a framework region 2 having the amino acid sequence set forth in SEQ ID NO:5 (or a variant of SEQ ID NO:5 with one, two, three, four, five, six, seven, eight, nine, ten, or more amino acid modifications), a framework region 3 having the amino acid sequence set forth in SEQ ID NO:6 (or a variant of SEQ ID NO:6 with one, two, three, four, five, six, seven, eight, nine, ten, or more amino acid modifications), and a framework region 4 having the amino acid sequence set forth in SEQ ID NO:7 (or a variant of SEQ ID NO:7 with one, two, three, four, five, six, seven, eight, nine, ten, or more amino acid modifications) and/or (b) a light chain variable domain that includes a framework region 1 having the amino acid sequence set forth in SEQ ID NO:11 (or a variant of SEQ ID NO:11 with one, two, three, four, five, six, seven, eight, nine, ten, or more amino acid modifications), a framework region 2 having the amino acid sequence set forth in SEQ ID NO:12 (or a variant of SEQ ID NO:12 with one, two, three, four, five, six, seven, eight, nine, ten, or more amino acid modifications), a framework region 3 having the amino acid sequence set forth in SEQ ID NO:13 (or a variant of SEQ ID NO:13 with one, two, three, four, five, six, seven, eight, nine, ten, or more amino acid modifications), and a framework region 4 having the amino acid sequence set forth in SEQ ID NO:14 (or a variant of SEQ ID NO:14 with one, two, three, four, five, six, seven, eight, nine, ten, or more amino acid modifications).

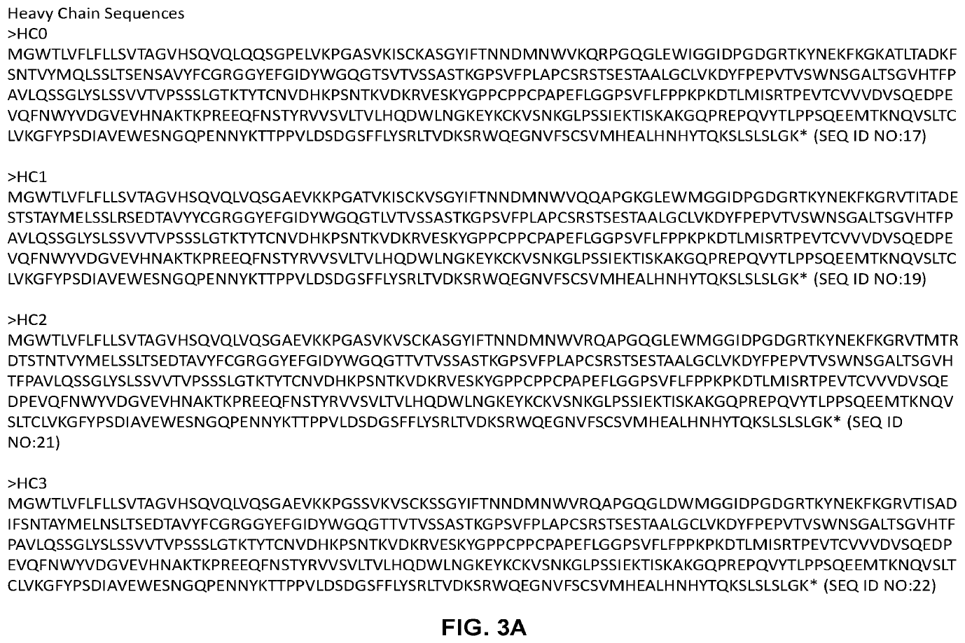

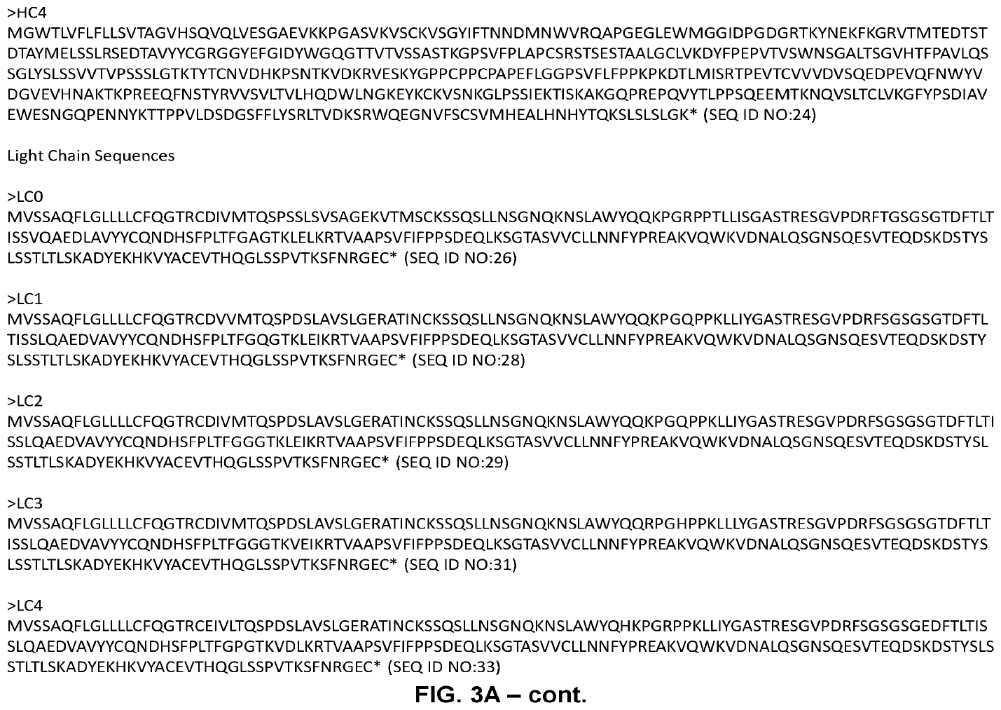

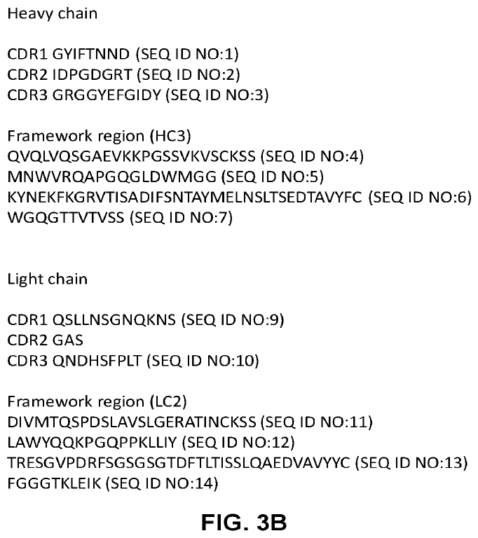

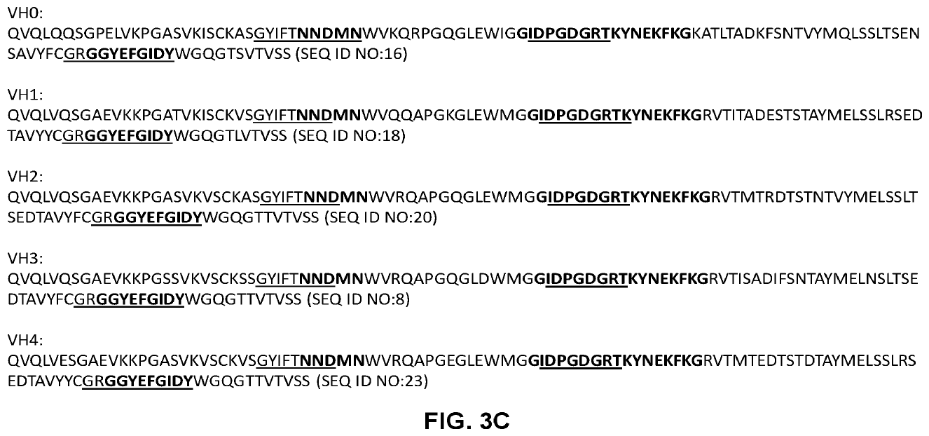

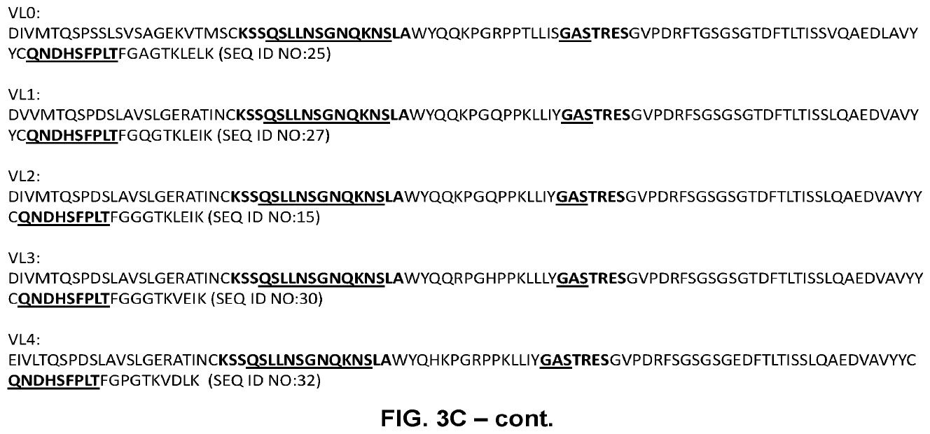

In some cases, an antigen binding domain of a CAR having any of the CDRs set forth in FIG. 3B can be designed to include framework regions (e.g., any of the framework regions of heavy chain variable region VH0 (SEQ ID NO:16), VH1 (SEQ ID NO: 18), VH2 (SEQ ID NO:20), VH3 (SEQ ID NO:8), or VH4 (SEQ ID NO:23) and/or any of the framework regions of light chain variable region LC0 (SEQ ID NO:25), LC1 (SEQ ID NO:27), LC2 (SEQ ID NO:15), LC3 (SEQ ID NO:30), or LC4 (SEQ ID NO: 32)) or can be designed to include one or more framework regions from another antibody, antibody fragment, or antibody domain.

In some cases, an antigen binding domain of a CAR provided herein having the ability to bind to a TRAILshort polypeptide (e.g., a human TRAILshort polypeptide) can include (a) a heavy chain variable domain that includes an amino acid sequence having at least 90 percent identity to the amino acid sequence set forth in SEQ ID NO:8 and/or (b) a light chain variable domain that includes an amino acid sequence having at least 90 percent identity to the amino acid sequence set forth in SEQ ID NO:15. For example, a CAR provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:8 and/or (b) a light chain variable domain that includes an amino acid sequence having at least 90, 91, 92, 93, 94, 95, 96, 97, 98, or 99 percent identity to the amino acid sequence set forth in SEQ ID NO: 15. In some cases, an antigen binding domain of a CAR provided herein can include (a) a heavy chain variable domain that includes an amino acid sequence having 100 percent identity to the amino acid sequence set forth in SEQ ID NO:8 and/or (b) a light chain variable domain that includes an amino acid sequence having 100 percent identity to the amino acid sequence set forth in SEQ ID NO:15.

In some cases, a CAR provided herein having the ability to bind to a TRAILshort polypeptide (e.g., a human TRAILshort polypeptide) can include an antigen binding domain that has (a) a heavy chain variable domain that includes an amino acid sequence having at least 90 percent identity to the amino acid sequence set forth in SEQ ID NO:8, provided that the heavy chain variable domain includes the amino acid sequences set forth in SEQ ID NOs: 1, 2, and 3, and/or (b) a light chain variable domain that includes an amino acid sequence having at least 90 percent identity to the amino acid sequence set forth in SEQ ID NO:15, provided that the light chain variable domain includes the amino acid sequences set forth in SEQ ID NO:9, the amino acid sequence GAS, and SEQ ID NO: 10. For example, an antigen binding domain of a CAR provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:8, provided that the heavy chain variable domain includes the amino acid sequences set forth in SEQ ID NOs: 1, 2, and 3, and/or (b) a light chain variable domain that includes an amino acid sequence having at least 90, 91, 92, 93, 94, 95, 96, 97, 98, or 99 percent identity to the amino acid sequence set forth in SEQ ID NO:15, provided that the light chain variable domain includes the amino acid sequences set forth in SEQ ID NO:9, the amino acid sequence GAS, and SEQ ID NO:10.

In some cases, an antigen binding domain of a CAR provided herein having the ability to bind to a TRAILshort polypeptide (e.g., a human TRAILshort polypeptide) can include (a) a heavy chain variable domain having the amino acid sequence set forth in SEQ ID NO:8 or the amino acid set forth in SEQ ID NO:8 with one, two, three, four, five, six, seven, eight, nine, or 10 amino acid modifications (e.g., amino acid substitutions, amino acid deletions, and/or amino acid additions) and/or (b) a light chain variable domain that includes the amino acid sequence set forth in SEQ ID NO:15 or the amino acid set forth in SEQ ID NO:15 with one, two, three, four, five, six, seven, eight, nine, or 10 amino acid modifications (e.g., amino acid substitutions, amino acid deletions, and/or amino acid additions). For example, an antigen binding domain of a CAR provided herein can have the ability to bind to a TRAILshort polypeptide (e.g., a human TRAILshort polypeptide), can include a heavy chain variable domain having the amino acid sequence set forth in SEQ ID NO:8 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs: 1, 2, and 3, and can include a light chain variable domain having the amino acid sequence set forth in SEQ ID NO:15 with one, two, three, four, five, six, seven, eight, nine, or 10 amino acid modifications (e.g., amino acid substitutions, amino acid deletions, and/or amino acid additions), provided that the light chain variable domain includes the amino acid sequences set forth in SEQ ID NO:9, the amino acid sequence GAS, and SEQ ID NO:10.

In some cases, a CAR provided herein having the ability to bind to a TRAILshort polypeptide (e.g., a human TRAILshort polypeptide) can include an antigen binding domain having (a) a heavy chain variable domain comprising (i) a CDR1 that comprises, consists essentially of, or consists of the amino acid sequence set forth in SEQ ID NO:1, (ii) a CDR2 that comprises, consists essentially of, or consists of the amino acid sequence set forth in SEQ ID NO:2, and (iii) a CDR3 that comprises, consists essentially of, or consists of the amino acid sequence set forth in SEQ ID NO:3, and/or (b) a light chain variable domain comprising (i) a CDR1 that comprises, consists essentially of, or consists of the amino acid sequence set forth in SEQ ID NO:9, (ii) a CDR2 that comprises, consists essentially of, or consists of the amino acid sequence GAS, and (iii) a CDR3 that comprises, consists essentially of, or consists of the amino acid sequence set forth in SEQ ID NO: 10.

As used herein, a “CDR1 that consists essentially of the amino acid sequence set forth in SEQ ID NO:1” is a CDR1 that has zero, one, or two amino acid substitutions within SEQ ID NO: 1, that has zero, one, two, three, four, or five amino acid residues directly preceding SEQ ID NO:1, and/or that has zero, one, two, three, four, or five amino acid residues directly following SEQ ID NO:1, provided that the CAR maintains its basic ability to bind to a TRAILshort polypeptide (e.g., a human TRAILshort polypeptide).

As used herein, a “CDR2 that consists essentially of the amino acid sequence set forth in SEQ ID NO:2” is a CDR2 that has zero, one, or two amino acid substitutions within SEQ ID NO:2, that has zero, one, two, three, four, or five amino acid residues directly preceding SEQ ID NO:2, and/or that has zero, one, two, three, four, or five amino acid residues directly following SEQ ID NO:2, provided that the CAR maintains its basic ability to bind to a TRAILshort polypeptide (e.g., a human TRAILshort polypeptide).

As used herein, a “CDR3 that consists essentially of the amino acid sequence set forth in SEQ ID NO:3” is a CDR3 that has zero or one amino acid substitutions within SEQ ID NO:3, that has zero, one, two, three, four, or five amino acid residues directly preceding SEQ ID NO:3, and/or that has zero, one, two, three, four, or five amino acid residues directly following SEQ ID NO:3, provided that the CAR maintains its basic ability to bind to a TRAILshort polypeptide (e.g., a human TRAILshort polypeptide).

As used herein, a “CDR1 that consists essentially of the amino acid sequence set forth in SEQ ID NO:9” is a CDR1 that has zero, one, or two amino acid substitutions within SEQ ID NO:9, that has zero, one, two, three, four, or five amino acid residues directly preceding SEQ ID NO:9, and/or that has zero, one, two, three, four, or five amino acid residues directly following SEQ ID NO:9, provided that the CAR maintains its basic ability to bind to a TRAILshort polypeptide (e.g., a human TRAILshort polypeptide).

As used herein, a “CDR2 that consists essentially of the amino acid sequence GAS” is a CDR2 that has zero or one amino acid substitution within the GAS sequence, that has zero, one, two, three, four, or five amino acid residues directly preceding the GAS sequence, and/or that has zero, one, two, three, four, or five amino acid residues directly following the GAS sequence, provided that the CAR maintains its basic ability to bind to a TRAILshort polypeptide (e.g., a human TRAILshort polypeptide).

As used herein, a “CDR3 that consists essentially of the amino acid sequence set forth in SEQ ID NO:10” is a CDR3 that has zero, one, or two amino acid substitutions within SEQ ID NO:10, that has zero, one, two, three, four, or five amino acid residues directly preceding SEQ ID NO:10, and/or that has zero, one, two, three, four, or five amino acid residues directly following SEQ ID NO:10, provided that the CAR maintains its basic ability to bind to a TRAILshort polypeptide (e.g., a human TRAILshort polypeptide).

When designing a single chain antibody (e.g., a scFv) having a heavy chain variable domain and a light chain variable domain, the two regions can be directly connected or can be connected using any appropriate linker sequence. For example, a heavy chain variable domain having the CDRs of SEQ ID NOs: 1-3 can be directly connected to a light chain variable domain having the CDRs of SEQ ID NO:9, the amino acid sequence GAS, and SEQ ID NO:11, respectively, via a linker sequence. An example of a linker sequence that can be used to connect a heavy chain variable domain and a light chain variable domain to create a scFv include, without limitation, (GGGGS) 3-5 (SEQ ID NOS: 47, 158, and 159, respectively). In some cases, for example, a linker that can be used to connect a heavy chain variable domain and a light chain variable domain to create a scFv can have the amino acid sequence GGGGSGGGGGGGGS (SEQ ID NO: 47), which can be encoded by the nucleic acid sequence ggtggcggtggctcgggcggtggtgggtcgggtggcggcggatct (SEQ ID NO:156).

As indicated herein, the amino acid sequences described herein can include amino acid modifications (e.g., the articulated number of amino acid modifications). Such amino acid modifications can include, without limitation, amino acid substitutions, amino acid deletions, amino acid additions, and combinations. In some cases, an amino acid modification can be made to improve the binding and/or contact with an antigen and/or to improve a functional activity of a CAR provided herein. In some cases, an amino acid substitution within an articulated sequence identifier can be a conservative amino acid substitution. For example, conservative amino acid substitutions can be made by substituting one amino acid residue for another amino acid residue having a similar side chain. Families of amino acid residues having similar side chains can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some cases, an amino acid substitution within an articulated sequence identifier can be a non-conservative amino acid substitution. Non-conservative amino acid substitutions can be made by substituting one amino acid residue for another amino acid residue having a dissimilar side chain. Examples of non-conservative substitutions include, without limitation, substituting (a) a hydrophilic residue (e.g., serine or threonine) for a hydrophobic residue (e.g., leucine, isoleucine, phenylalanine, valine, or alanine); (b) a cysteine or proline for any other residue; (c) a residue having a basic side chain (e.g., lysine, arginine, or histidine) for a residue having an acidic side chain (e.g., aspartic acid or glutamic acid); and (d) a residue having a bulky side chain (e.g., phenylalanine) for glycine or other residue having a small side chain.

Methods for generating an amino acid sequence variant (e.g., an amino acid sequence that includes one or more modifications with respect to an articulated sequence identifier) can include site-specific mutagenesis or random mutagenesis (e.g., by PCR) of a nucleic acid encoding the antibody or fragment thereof. See, for example, Zoller, Curr. Opin. Biotechnol. 3:348-354 (1992). Both naturally occurring and non-naturally occurring amino acids (e.g., artificially-derivatized amino acids) can be used to generate an amino acid sequence variant provided herein.

A representative number of antigen binding domains having the ability to bind to a TRAILshort polypeptide (e.g., a human TRAILshort polypeptide) are further described in Table 1.

TABLE 1
Representative number of antigen binding domains.

| | | SEQ ID NOs of | | | SEQ ID NOs of | |
|---|---|---|---|---|---|---|
| | SEQ ID NOs of | Heavy Chain | | SEQ ID NOs of | Light Chain | |
| | Heavy Chain | Variable | SEQ ID NO of | Light Chain | Variable | SEQ ID NO of |
| | Variable | Domain/Region | Heavy Chain | Variable | Domain/Region | Light Chain |
| | Domain/Region | Framework | Variable | Domain/Region | Framework | Variable |
| Antibody | CDRs | Regions | Domain/Region | CDRs | Regions | Domain/Region |
| Ab866 | 1, 2, 3 | 4, 5, 6, 7 | 8 | 9, GAS, 10 | 11, 12, 13, 14 | 15 |

GAS = Glycine-Alanine-Serine

The CARs provided herein can be produced using any appropriate method. For example, the CARs provided herein can be produced in recombinant host cells. For example, a nucleic acid encoding a CAR provided herein can be constructed, introduced into an expression vector, and expressed in suitable host cells. In some cases, a binder (e.g., an antibody, antigen binding fragment, antibody domain, and/or CAR) provided herein can be recombinantly produced in prokaryotic hosts such as E. coli, Bacillus brevis, Bacillus subtilis, Bacillus megaterium, Lactobacillus zeae/casei, or Lactobacillus paracasei. A binder (e.g., an antibody, antigen binding fragment, antibody domain, and/or CAR) provided herein also can be recombinantly produced in eukaryotic hosts such as yeast (e.g., Pichia pastoris, Saccharomyces cerevisiae, Hansenula polymorpha, Schizosaccharomyces pombe, Schwanniomyces occidentalis, Kluyveromyces lactis, or Yarrowia lipolytica), filamentous fungi of the genera Trichoderma (e.g., T. reesei) and Aspergillus (e.g., A. niger and A. oryzae), protozoa such as Leishmania tarentolae, insect cells, or mammalian cells (e.g., mammalian cell lines such as Chinese hamster ovary (CHO) cells, Per.C6 cells, mouse myeloma NS0 cells, baby hamster kidney (BHK) cells, or human embryonic kidney cell line HEK293). See, for example, the Frenzel et al. reference (Front Immunol., 4:217 (2013)).

In some cases, an antigen binding fragment or antibody domain provided herein can be produced by proteolytic digestion of an intact antibody. For example, an antigen binding fragment can be obtained by treating an antibody with an enzyme such as papain or pepsin. Papain digestion of whole antibodies can be used to produce F(ab)₂ or Fab fragments, while pepsin digestion of whole antibodies can be used to produce F(ab′)₂ or Fab′ fragments.

In some cases, a CAR provided herein can be substantially pure. The term “substantially pure” as used herein with reference to a CAR refers to the CAR as being substantially free of other polypeptides, lipids, carbohydrates, and nucleic acid with which it is naturally associated. Thus, a substantially pure CAR provided herein is any CAR that is removed from its natural environment and is at least 60 percent pure. A substantially pure CAR provided herein can be at least about 65, 70, 75, 80, 85, 90, 95, or 99 percent pure.

This document also provides nucleic acid molecules (e.g., isolated nucleic acid molecules) having a nucleic acid sequence encoding at least part of a CAR provided herein. For example, an isolated nucleic acid molecule provided herein can include a nucleic acid sequence encoding a heavy chain variable domain such as a heavy chain variable domain as set forth in FIG. 3C. In another example, an isolated nucleic acid molecule provided herein can include a nucleic acid sequence encoding a light chain variable domain such as a light chain variable domain as set forth in FIG. 3C. In some cases, an isolated nucleic acid molecule provided herein can include a nucleic acid sequence encoding both (a) a heavy chain variable domain and (b) a light chain variable domain, with or without, encoding a linker polypeptide (e.g., (SGGGG)₃₋₅). A nucleic acid provided herein (e.g., an isolated nucleic acid molecule) can be single stranded or double stranded nucleic acid of any appropriate type (e.g., DNA, RNA, or DNA/RNA hybrids).

This document also provides vectors (e.g., plasmid vectors or viral vectors) containing one or more nucleic acids provided herein. An example of a plasmid vector that can be designed to include one or more nucleic acids having a nucleic acid sequence encoding at least part of a CAR provided herein includes, without limitation, phagemids and viral vectors. Examples of viral vectors that can be designed to include one or more nucleic acids having a nucleic acid sequence encoding at least part of a CAR provided herein include, without limitation, retroviral vectors, parvovirus-based vectors (e.g., adenoviral-based vectors and adeno-associated virus (AAV)-based vectors), lentiviral vectors (e.g., herpes simplex (HSV)-based vectors), poxviral vectors (e.g., vaccinia virus-based vectors and fowlpox virus-based vectors), and hybrid or chimeric viral vectors. For example, a viral vector having an adenoviral backbone with lentiviral components such as those described elsewhere (Zheng et al., Nat. Biotech., 18(2): 176-80 (2000); WO 98/22143; WO 98/46778; and WO 00/17376) or viral vectors having an adenoviral backbone with AAV components such as those described elsewhere (Fisher et al., Hum. Gene Ther., 7:2079-2087 (1996)) can be designed to include one or more nucleic acids having a nucleic acid sequence encoding at least part of a CAR provided herein.

In some cases, a vector (e.g., a plasmid vector or a viral vector) provided herein can include a nucleic acid sequence encoding an scFv or antibody binding domain provided herein. In some cases, a vector (e.g., a plasmid vector or a viral vector) provided herein can include a nucleic acid sequence encoding a CAR provided herein.

A vector provided herein (e.g., a plasmid vector or viral vector provided herein) can include any appropriate promoter and other regulatory sequence (e.g., transcription and translation initiation and termination codons) operably linked the nucleic acid sequence encoding at least part of a CAR provided herein. In some cases, a promoter used to drive expression can be a constitutive promotor or a regulatable promotor. Examples of regulatable promoters that can be used as described herein include, without limitation, inducible promotors, repressible promotors, and tissue-specific promoters. Examples of viral promotors that can be used as described herein include, without limitation, adenoviral promotors, vaccinia virus promotors, CMV promotors (e.g., immediate early CMV promotors), and AAV promoters.

Any appropriate method can be used to make a nucleic acid molecule (or vector such as a plasmid vector or viral vector) having a nucleic acid sequence encoding at least part of a CAR provided herein. For example, molecule cloning techniques can be used to make a nucleic acid molecule (or vector such as a plasmid vector or viral vector) having a nucleic acid sequence encoding at least part of a CAR provided herein as described elsewhere (see, e.g., Sambrook et al., Molecular Cloning: A Laboratory Manual, 2nd edition, Cold Spring Harbor Laboratory, NY (1989); and Ausubel et al., Current Protocols in Molecular Biology, Green Publishing Associates and John Wiley & Sons, New York, N.Y. (1994)).

This document also provides host cells that include a nucleic acid provided herein (e.g., a nucleic acid having a nucleic acid sequence encoding at least part of a CAR). Host cells that can be designed to include one or more nucleic acids provided herein can be prokaryotic cells or eukaryotic cells. Examples of prokaryotic cells that can be designed to include a nucleic acid provided herein include, without limitation, E. coli (e.g., Tb-1, TG-1, DH5a, XL-Blue MRF (Stratagene), SA2821, or Y1090 cells), Bacillus subtilis, Salmonella typhimurium, Serratia marcescens, or Pseudomonas (e.g., P. aerugenosa) cells. Examples of eukaryotic cells that can be designed to include a nucleic acid provided herein include, without limitation, insect cells (e.g., Sf9 or Ea4 cells), yeast cells (e.g., S. cerevisiae cells), and mammalian cells (e.g., mouse, rat, hamster, monkey, or human cells). For example, VERO cells, HeLa cells, 3T3 cells, Chinese hamster ovary (CHO) cells, W138 BHK cells, COS-7 cells, and MDCK cells can be designed to include a nucleic acid provided herein. Any appropriate method can be used to introduce one or more nucleic acids provided herein (e.g., a vector such as a plasmid vector or viral vector having a nucleic acid sequence encoding at least part of a binder provided herein) into a host cell. For example, calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, electroporation, or combinations thereof can be used to introduce a nucleic acid provided herein into a host cell (see, e.g., Sambrook et al., Molecular Biology: A Laboratory Manual, Cold Spring Harbor Laboratory, NY (1989); Davis et al., Basic Methods in Molecular Biology (1986); and Neumann et al., EMBO J., 1:841 (1982)).

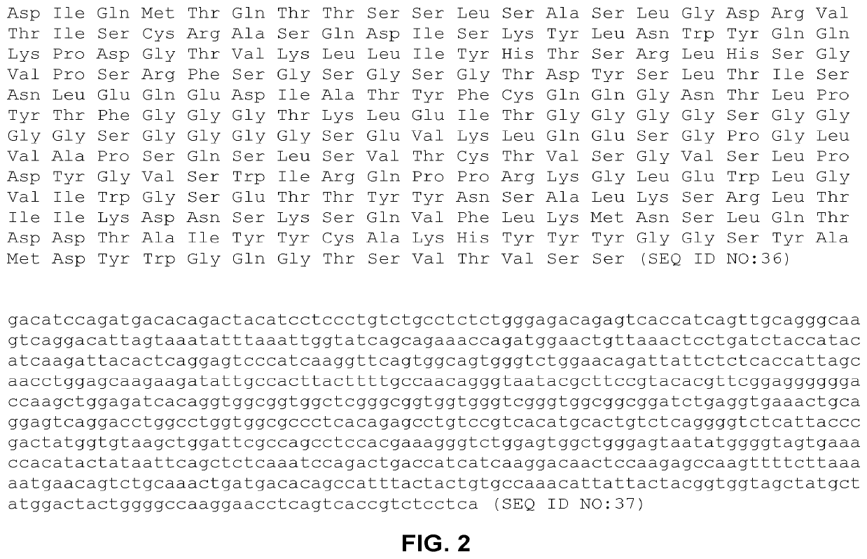

In some cases, cells such as T cells, stem cells (e.g., induced pluripotent stem cells or mesenchymal stem cells), or NK cells can be designed to express one or more nucleic acids encoding a CAR described herein. In some cases, at least 50 percent, at least 75 percent, at least 95 percent, at least 99 percent, or 100 percent of the cells express the one or more CARs. For example, a population of T cells can be infected with viral vectors designed to express nucleic acid encoding a CAR described herein (e.g., a CAR having the ability to bind to a TRAILshort polypeptide). In some embodiments, a population of T cells also can be designed to express a second CAR, e.g., a CAR having the ability to bind to any one of a human CD19 polypeptide, a human BCMA polypeptide, a human TSHR polypeptide, a human EPHA3 polypeptide, a human FGFR2 polypeptide, a human HER2 polypeptide, a human TROP2 polypeptide, a human NY-ESO polypeptide, a human Mesothelin polypeptide, a human EGFR polypeptide, a human EGFRviii polypeptide, a human IL13Ra2 polypeptide, a human Folate receptor alpha polypeptide, a human Folate receptor Beta polypeptide, a human gut integrin (e.g., ITGB7) polypeptide, a human CD103 polypeptide, a human CD83 polypeptide, a human CD22 polypeptide, a human CD20 polypeptide, a human CD79b polypeptide, a human CD79a polypeptide, a human CD123 polypeptide, a human CD33 polypeptide, a human ILR1a polypeptide, a human CD34 polypeptide, a human CD30 polypeptide, a human CD4 polypeptide, a human CD8 polypeptide, a human T cell receptor alpha polypeptide, a human T cell receptor beta polypeptide, a human CD3 polypeptide, a human CD5 polypeptide, a human CD7 polypeptide, a human gp120 polypeptide, a human galactomannan polypeptide, a human PSMA polypeptide, a human MUC polypeptide, a human PD-1 polypeptide, a human CD80 polypeptide, a human CD86 polypeptide, a human CEA polypeptide, a human GPC3 polypeptide, a human ROR1 polypeptide, a human AFP polypeptide, a human CD138 polypeptide, a human CD38 polypeptide, a human CD44v6 polypeptide, a human CD70 polypeptide, a human CLEC12A (CLL-1) polypeptide, a human CS-1 polypeptide, a human FAP polypeptide, a human GPRC5D polypeptide, a human MUC-1 polypeptide, a human MUC16 polypeptide, a human NKG2D polypeptide, or any other target that complements the TRAILshort activity. For example, a CAR having the ability to bind to a human CD19 polypeptide can include an antigen binding domain, a hinge, a transmembrane domain, and one or more signaling domains. An antigen binding domain of such a CAR can include the CDRs of an FMC63 or 4G7 scFv. In some cases, the antigen binding domain of such a CAR can include an FMC63 or 4G7 scFv. See, e.g., Kang et al., Int. J. Mol. Sci., 21(23):9163 (2020) and U.S. U.S. Pat. No. 9,499,629. See also FIG. 2 for the amino acid sequence of the FMC63 scFv and the nucleic acid sequence encoding the scFv. The hinge of the second CAR can be a hinge set forth in FIG. 7A, the transmembrane domain of the second CAR can be a transmembrane domain set forth in FIG. 7C, and the one or more signaling domains of the second CAR can be selected from the group of signaling domains set forth in FIG. 7D. For example, a CAR having the ability to bind to a human CD19 polypeptide can include a CD8a leader, a CD8a hinge, a CD8a transmembrane domain, a 4-1BB intracellular signaling domain, and a CD3 ζ intracellular signaling domain.

Examples of scFv sequences and other binding sequences that can be used to design a CAR to a particular tumor antigen include, without limitation, those set forth in Table 2.

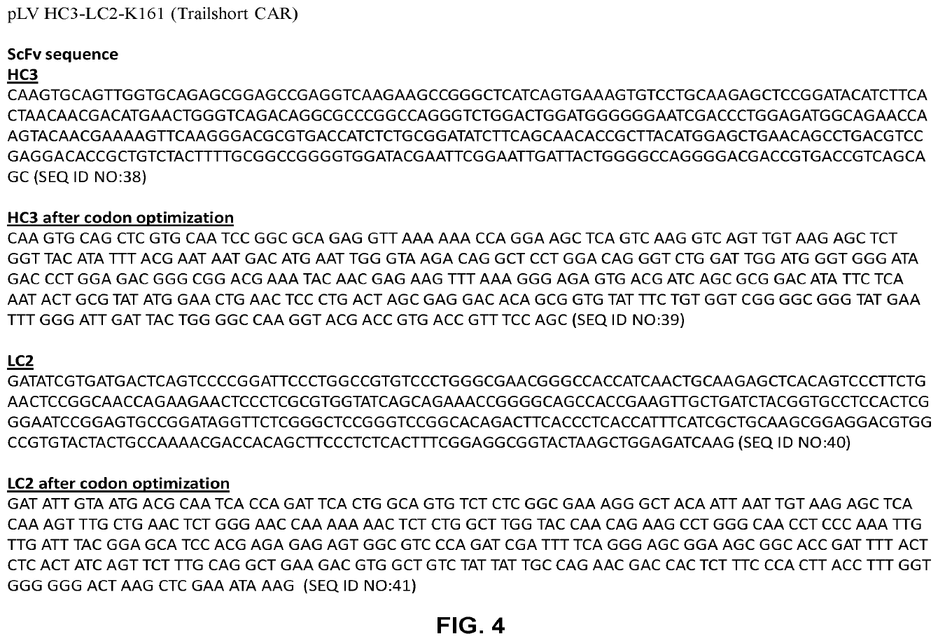

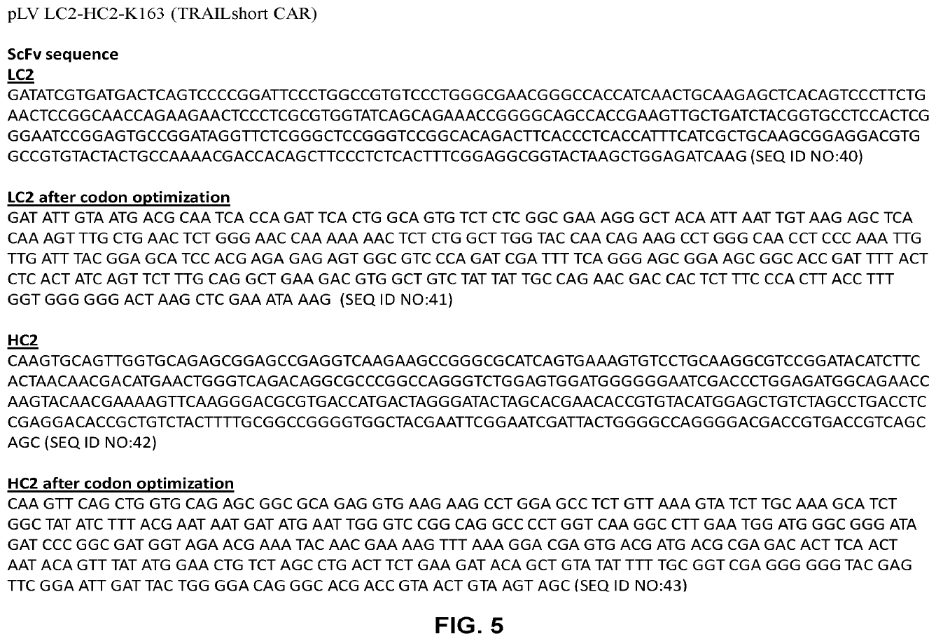

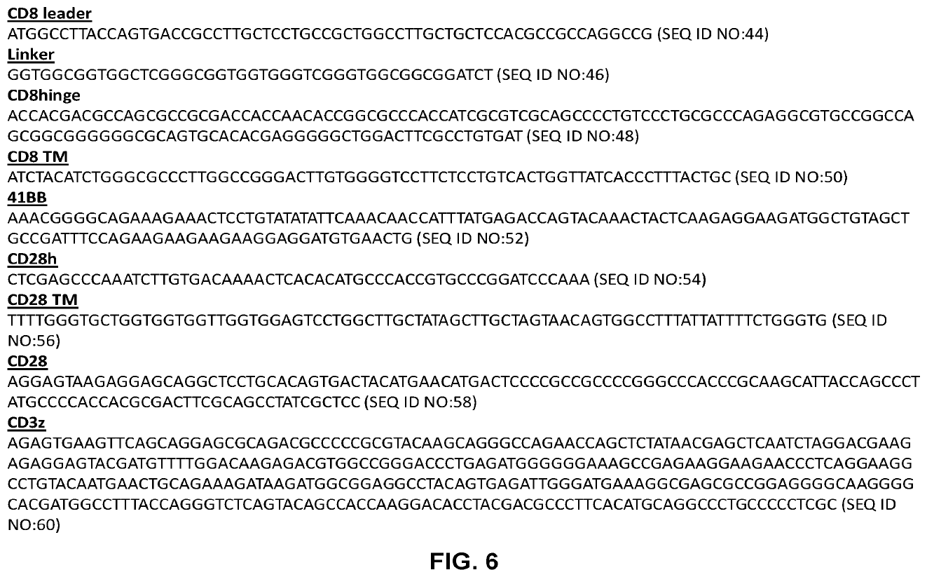

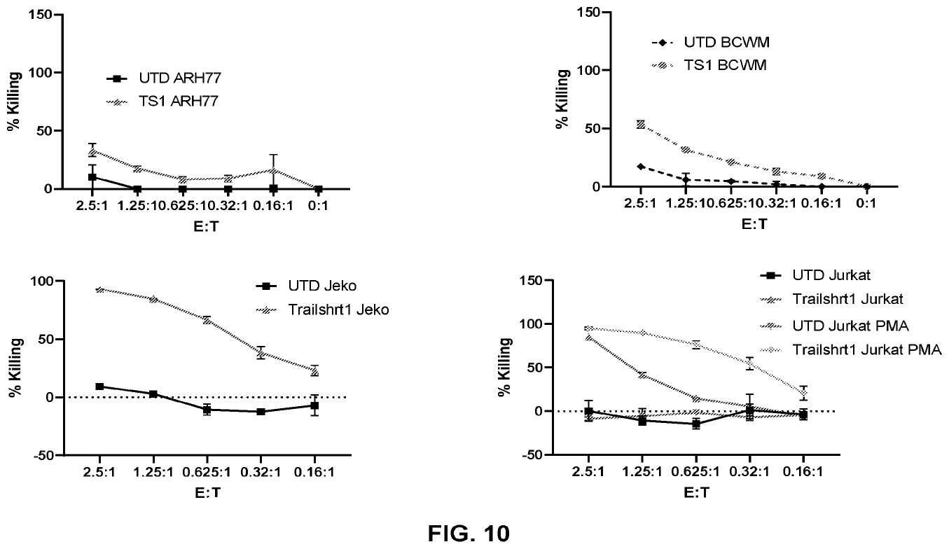

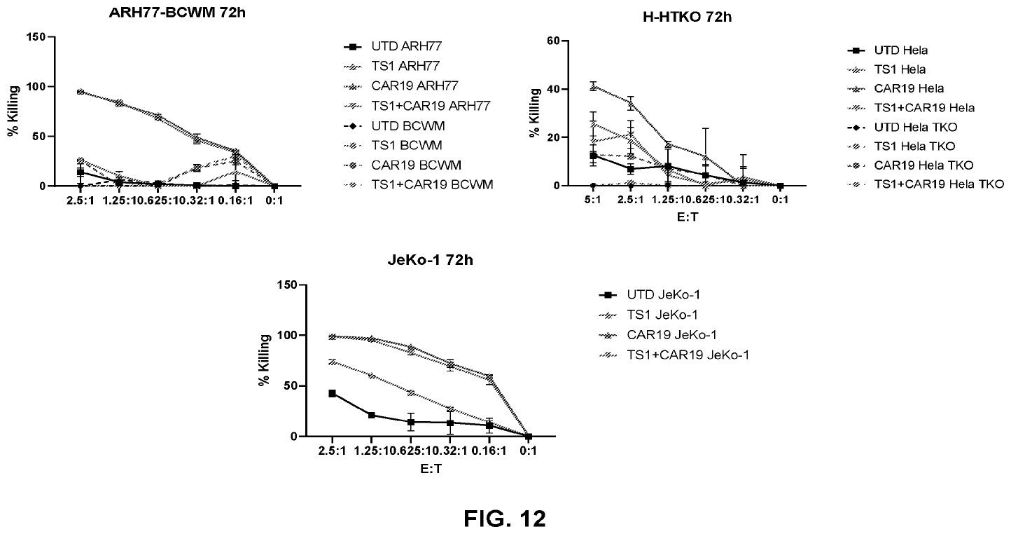

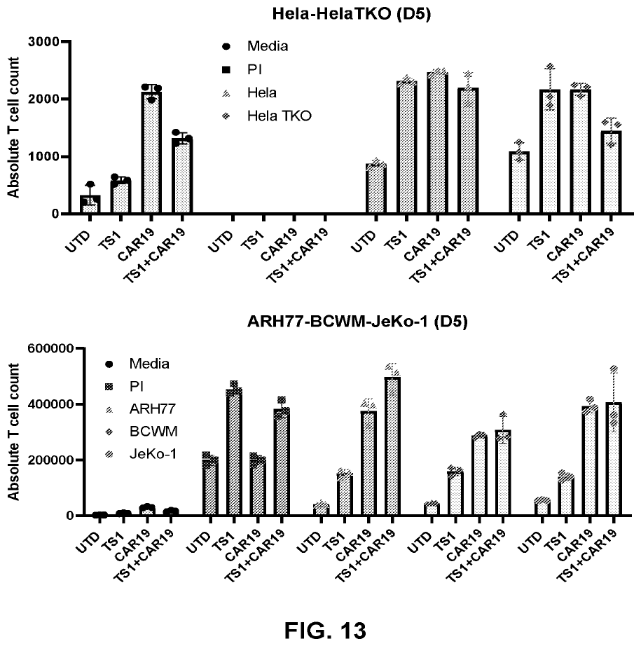

TABLE 2
Representative scFv antibodies and sequences for targeting CARs to tumor antigens

| Antigen | scFv antibody name | Reference(s) |
|---|---|---|
| CD19 | FMC63 | Milone et al., Mol. Ther., 17(8): 1453-64 (2009) (PMID: 26330164) |
| | MOR208 | Kellner et al., Leukemia, 27(7): 1595-8 (2013) (PMID: 23277329) |
| | Humanized scFv | WO2015/157252; see, e.g., Tables 4 and 5. |
| | 4G7 | EP Patent No. EP2997141 |
| | Low affinity scFv | Chinese Patent No. CN107406517; see, e.g., Abstract |
| MUC-1 | 5E5 | Posey et al., Immunity, 45(5): 947-948 (2016) (PMID: 27851918) |
| HER-2 | 4D5 | Ohnishi et al., Br. J. Cancer, 71(5): 969-73 (1995) (PMID: 7734322) |
| | | Forsberg et al., Cancer Res., 79(5): 899-904 (2019) (PMID: 30622115) |
| | | Nellan et al., J. Immunother. Cancer, 6(1): 30 (2018) (PMID: 29712574) |
| | | Priceman et al., Clin Cancer Res., 24(1): 95-105 (2018) (PMID: 29061641) |
| | FRP5 | Bielamowics et al., Neuro. Oncol., 20(4): 506-518 (2018) (PMID: 29016929) |
| EGFR | M27 | Jiang et al., Cancer Immunol. Res., 6(11): 1314-1326 (2018) (PMID: 30201736) |
| | Cetuximab | Caruso et al., Cancer Res., 75(17): 3505-18 (2015) (PMID: 26330164) |
| Folate receptor alpha | C4 based | Ao et al., J. Immunother., 42(8): 284-296 (2019) (PMID: 31261167) |
| | MOv19 | Song et al., J. Hematol. Oncol., 9(1): 56 (2016) (PMID: 27439908) |
| Mesothelin | SS1 | Haas et al., Mol. Ther., S1525-0016(19)30328-4 (2019) (PMID: 31420241) |
| | M clone | Adusumilli et al., Sci. Transl. Med., 6(261): 261ra151 (2014) (PMID: 25378643) |
| | Amatuximab | WO2022/082068A1; see, e.g., Paragraph 106 |
| | Anetumab | U.S. Pat. No. 9,023,351B2; see, e.g., Claim 1 |
| AFP | ET1402L1 | Liu et al., Clin. Cancer Res., 23(2): 478-488 (2017) (PMID: 27535982) |
| CEA | Anti-CEA scFv | Chi et al., Cancer Med., 8(10): 4753-4765 (2019) (PMID: 31237116) |
| | CEACAMS | Thistlethwaite et al., Cancer Immunol. Immunother., 66(11): 1425-1436 (2017) |
| | | (PMID: 28660319) |
| | hMN14 | Katz et al., Clin. Cancer Res., 21(14): 3149-59 (2015) (PMID: 25850950) |
| CD123 | 22172, 22176 | Gill et al., Blood, 123(15): 2343-54 (2014) (PMID: 24596416) |
| | Humanized scFv | US 2016/0068601 |
| | Humanized scFv | EP Patent No. EP2968415 |
| | Tagrazofusp based | Pemmaraju et al. N. Engl. J. Med., 380(17): 1628-1637 (2019) (PMID: 31018069) |
| CD33 | MY96 | Kenderian et al., Leukemia, 29(8): 1637-47 (2015) (PMID: 25721896) |
| | Humanized scFv | WO2016/014576 |
| CLEC12A (CLL-1) | M6E7 | US 2020/0148774A1; see, e.g., FIG. 1 |
| | m21C9 | US 2020/0148774A1; see, e.g., FIG. 1 |
| | M20B1 | US 2020/0148774A1; see, e.g., FIG. 1 |
| | M28H12 | US 2020/0148774A1; see, e.g., FIG. 1 |
| | Humanized scFv | US 2016/0051651 |
| | CLL1 scFv | WO2016120219 |
| CD22 | M0971 | Fry et al., Nat. Med., 24(1): 20-28 (2018) (PMID: 29155426) |
| | Humanized scFv clones | WO2016/164731 |
| | Inotuzumab based | Kantarjian et al., N. Engl. J. Med., 375(8): 740-53 (2016) (PMID: 27292104) |
| | Moxetumomab based | Kreitman et al., Leukemia, 32(8): 1768-1777 (2018) (PMID: 30030507) |
| CD20 | Rituximab | Zhang et al., Signal Transduct. Target Ther., 1: 16002 (2016) (PMID: 29263894) |
| | Leu 16 | Lee et al., J. Immunother., 41(1): 19-31 (2018) (PMID: 29176334) |
| | CD20 scFvs | WO2016/164731 |
| BCMA | BCMA-02 | Raje et al., N. Engl. J. Med., 380(18): 1726-1737 (2019) (PMID: 31042825) |
| | LCAR38 | Zhao et al., J. Hematol. Oncol., 11(1): 141 (2018) (PMID: 30572922) |
| | BCMA scFv | Smith et al., Cancer Immunol. Res., 7(7): 1047-1053 (2019) (PMID: 31113804) |
| | Biepitopic | Xu et al., Proc. Natl. Acad. Sci. USA, 116(19): 9543-9551 (2019) (PMID: |
| | | 30988175) |
| | NVS BCMA | Cohen et al., J. Clin. Invest., 129(6): 2210-2221 (2019) (PMID: 30896447) |
| CS-1 | CS1R | Wang et al., Clin. Cancer Res., 24(1): 106-119 (2018) (PMID: 29061640) |
| | CS1 ScFv | Chu et al., Leukemia, 28(4): 917-27 (2014) (PMID: 24067492) |
| | Elotuzumab based | Dimopoulos et al., N. Engl. J. Med., 379(19): 1811-1822 (2018) (PMID: 30403938) |
| CD138 | ScFv | Sun et al., Oncotarget., 10(24): 2369-2383 (2019) (PMID: 31040928) |
| CD44v6 | Humanized scFv | Leuci et al., Oncoimmunology, 7(5): e1423167 (2018) (PMID: 29721373) |
| | cMAb U36 | Sandstrom et al., Int. J. Oncol., 40(5): 1525-32 (2012) (PMID: 22307465) |
| NKG2D | scFv | Yang et al., J. Immunother. Cancer, 7(1): 171 (2019) (PMID: 31288857) |
| | NKG2Dg scFv | Parihar et al., Cancer Immunol. Res., 7(3): 363-375 (2019) (PMID: 30651290) |
| CD38 | Nanobody CD38 | An et al., Mol. Pharm., 15(10): 4577-4588 (2018) (PMID: 30185037) |
| | Humanized scFv | Yoshida et al., Clin. Transl. Immunology, 5(12): el16 (2016) (PMID: 28090317) |
| GPRC5D | Humanized scFv | Smith et al., Sci. Transl. Med., 11(485)eaau7746 (2019) (PMID: 30918115) |
| CD79b | scFv (L-H) and (H-L) | Ormhoj et al., Clin. Cancer Res., (2019) (PMID: 31439577) |
| CD103 | M290 | Zhang et al., Am. J. Transplant., 9(9): 2012-23 (2009) (PMID: 19645708) |
| FAP | FAP5 | Wang et al., Cancer Immunol. Res., 2(2): 154-66 (2014) (PMID: 24778279) |
| CD70 | Human CD70 | Park et al., Oral Oncol., 78: 145-150 (2018) (PMID: 29496042) |
| MUC16 | 4H11 | Koneru et al., Oncoimmunol., 4(3): e994446 (2015) (PMID: 25949921) |
| IL13Ra2 | IL13R | Kong et al., Clin. Cancer Res., 18(21): 5949-60 (2012) (PMID: 22966020) |
| CD3 | Muromonab | Kipriyanov et al., Protein Eng., 10(4): 445-453 (1997) (PMID: 9194170); see, e.g., |
| | | FIG. 2 |
| | Teplizumab | US 2007/0077246A1; see, e.g., FIG. 1 |
| | Blinatumomab | U.S. Pat. No. 8,076,459B2; see, e.g., FIG. 2 |
| CD30 | Brentuximab | Zhang et al., Mol. Pharm., 17(7): 2555-2569 (2020) (PMID: 32453957); see, e.g., |
| | | Table S1 |
| CD34 | 4C8 | Hou et al., J. Biochem., 144(1): 115-120 (PMID: 18424812) |
| CD4 | Ibalizumab | Zhang et al., Mol. Pharm., 17(7): 2555-2569 (2020) (PMID: 32453957); see, e.g., |
| | | Table S1 |
| CD5 | Anti-CD5 | WO2008/121160A3; see, e.g., Paragraph 0043 |
| | 9F2A11 | WO2022/040608A1; see, e.g., FIG. 4 |
| | Ab5D7v | US 2021/0355230A1; see, e.g., Paragraphs 0177-0180 |
| CD7 | Polatuzumab | Zhang et al., Mol. Pharm, 17(7): 2555-2569 (2020) (PMID: 32453957); see, e.g., |
| | | Table S1 |
| CD79a | Ab 4450 | U.S. Pat. No. 10,774,152B2; see, e.g., Paragraph 0725 |
| | Anti-CD79a | US 2021/0261659A1; see, e.g., Paragraphs 0158-0160 |
| CD8 | Crefmirlimab | US 2020/0140550A1; see, e.g., FIG. 2 |
| CD80 | Galiximab | Zhang et al., Mol. Pharm., 17(7): 2555-2569 (2020) (PMID: 32453957); see, e.g., |
| | | Table S1 |
| CD83 | 3C12 | U.S. Pat. No. 9,840,559B2; see, e.g., Claim 1 |
| CD86 | 32A | WO2017/105091A1; see, e.g., SEQ ID NOS: 4, 5, 6, 17, 18, and 19 therein |
| EGFRviii | Anti-EGFRvIII | U.S. Pat. No. 9,394,368B2; see, e.g., Example 4 |
| EPHA3 | Ifabotuzumab | WO2021/092560A1; see, e.g., Paragraph 0009 |
| FGFR2 | Aprutumab | WO2021/247718A1; see, e.g., Paragraph 0045 |
| | Bemarituzumab | US 2020/0347141A1; see, e.g., FIG. 13 |
| Folate receptor Beta | M909 | US 2021/0275684A1; see, e.g., FIG. 2 |
| | ASO4498 | US 2021/0275684A1; see, e.g., FIG. 3 |
| | Antibody #2 | US 2021/0275684A1; see, e.g., FIG. 4 |
| | Antibody #3 | US 2021/0275684A1; see, e.g., FIG. 5 |
| galactomannan | EH7 | CN111925993B; see, e.g., SEQ ID NOS: 1-6 therein |
| | BB10 | CN111925446B; see, e.g., SEQ ID NO: 1-6 therein |
| gp120 | Elipovimab | WO2018/237148A1; see, e.g., Table 1 |
| | Suvizumab | U.S. Pat. No. 6,114,143A; see, e.g., FIGS. 19 and 20 |
| | Teropavimab | U.S. Pat. No. 11,168,130B2; see, e.g., Paragraph 0017 |
| GPC3 | Codrituzumab | Zhang et al., Mol. Pharm., 17(7): 2555-2569 (2020) (PMID: 32453957); see, e.g., |
| | | Table S1 |
| gut integrins (ITGB7) | Etrolizumab | Zhang et al., Mol. Pharm, 17(7): 2555-2569 (2020) (PMID: 32453957); see, e.g., |
| | | Table S1 |
| ILR1a | 10E12 | WO2021/219872A1; see, e.g., Paragraphs 0047-0124 |
| | 9E11 | WO2021/219872A1; see, e.g., Paragraphs 0047-0124 |
| | 9G5 | WO2021/219872A1; see, e.g., Paragraphs 0047-0124 |
| MUC | Cantuzumab | WO2008/073598A2; see, e.g., FIG. 5 |
| | Clivatuzumab | US 2005/0014207A1; see, e.g., FIG. 1 |
| | Gatipotuzumab | U.S. Pat. No. 9,217,038B2; see, e.g., SEQ ID NOS: 1, 3, 5, 17, 19, and 21 therein |
| | Sofituzumab | US 2019/0336615A1; see, e.g., SEQ ID NOS: 117, 118, 119, 122, 123, and 124 |
| | | therein |
| | Ubamatamab | US 2020/0317810A1; see, e.g., SEQ ID NOS: 20, 22, 24, 28, 30, and 32 therein; |
| | | and Table 1 |
| NY-ESO (CTAG1B) | 12D7 | U.S. Pat. No. 8,519,106B2; see, e.g., FIG. 4 |
| | T1 | EP 2408819A2; see, e.g., FIG. 10 |
| | T2 | EP 2408819A2; see, e.g., FIG. 12 |
| | T3 | EP 2408819A2; see, e.g., FIG. 13 |
| PD-1 | Nivolumab | Zhang et al., Mol. Pharm., 17(7): 2555-2569 (2020) (PMID: 32453957); see, e.g., |
| | | Table S1 |
| | Pembrolizumab | Zhang et al., Mol. Pharm., 17(7): 25-2569 (2020) (PMID: 32453957); see, e.g., |
| | | Table S1 |
| PSMA | J591 | U.S. Pat. No. 10,179,819B2; see, e.g., SEQ ID NOS: 124, 126, 128, 932, 934, and 936 therein |
| ROR1 | Zilovertamab | US 2022/0133901A1; see, e.g., Table 1 |
| T cell receptor alpha | Anti-TCRa | U.S. Pat. No. 10,633,445B2; see, e.g., Paragraph 0008 |
| T cell receptor beta | Anti-TCRBC1 | US 2017/0334998A1; see, e.g., Paragraphs 0038-0043 |
| TSHR | K1-18 | U.S. Pat. No. 10,428,153B2; see, e.g., FIGS. 3B and 4B |
| TROP2 | Sacituzumab | US 2019/0336615A1; see, e.g., SEQ ID NOS: 195, 196, 197, 200, 201, and 202 |
| | Datopotamab | WO2021/242935A1; see, e.g., Paragraphs 0357-0358 |

In some cases, an antibody (e.g., an scFv antibody) that binds to an antigen described herein (e.g., an antigen listed in Table 2 such as CD19) can be designed to include the CDRs of a specific antibody described herein (e.g., the CDRs of a FMC63 scFv). For example, a scFv antibody can be designed to include the CDRs (e.g., CDR1, CDR2, and CDR3 of the heavy chain region and CDR1, CDR2, and CDR3 of the light chain region) of a FMC63 scFv with the same framework regions as the FMC63 scFv or with different framework regions, provided that the designed scFv antibody retains an ability to bind to a CD19 antigen. In some cases, a scFv antibody can be designed to include the CDRs (e.g., CDR1, CDR2, and CDR3 of the heavy chain region and CDR1, CDR2, and CDR3 of the light chain region) of an antibody (e.g., a scFv set forth in Table 2) with the framework regions being human framework regions, provided that the designed scFv antibody retains an ability to bind to its antigen.

In some cases, a nucleic acid provided herein (e.g., nucleic acid encoding a CAR provided herein), a vector provided herein (e.g., a viral vector designed to express a CAR provided herein), and/or a host cell provided herein (e.g., a host cell designed to express a CAR provided herein) can be formulated as a composition for administration to a mammal (e.g., a human) having cancer (e.g., a TRAILshort⁺ cancer) or a mammal (e.g., a human) having an infection to treat that mammal. In some cases, a nucleic acid provided herein (e.g., a nucleic acid encoding a CAR provided herein), a vector provided herein (e.g., a viral vector designed to express a CAR provided herein), and/or a host cell provided herein (e.g., a host cell designed to express one or more CARs provided herein) can be formulated as a pharmaceutical composition for administration to a mammal (e.g., a human) to reduce the number of cancer cells or infected cells within the mammal and/or to increase the survival of the mammal suffering from cancer (e.g., a TRAILshort⁺ cancer). In some cases, a pharmaceutical composition provided herein can include a pharmaceutically acceptable carrier such as a buffer, a salt, a surfactant, a sugar, a tonicity modifier, or combinations thereof as, for example, described elsewhere (Gervasi et al., Eur. J. Pharmaceutics and Biopharmaceutics, 131:8-24 (2018)). Examples of pharmaceutically acceptable carriers that can be used to make a pharmaceutical composition provided herein include, without limitation, water, lactic acid, citric acid, sodium chloride, sodium citrate, sodium succinate, sodium phosphate, a surfactant (e.g., polysorbate 20, polysorbate 80, or poloxamer 188), dextran 40, or a sugar (e.g., sorbitol, mannitol, sucrose, dextrose, or trehalose), or combinations thereof. For example, a pharmaceutical composition designed to include a CAR provided herein (or a nucleic acid, a vector, or a host cell provided herein) can be formulated to include a buffer (e.g., an acetate, citrate, histidine, succinate, phosphate, or hydroxymethylaminomethane (Tris) buffer), a surfactant (e.g., polysorbate 20, polysorbate 80, or poloxamer 188), and a sugar such as sucrose. Other ingredients that can be included within a pharmaceutical composition provided herein include, without limitation, amino acids such as glycine or arginine, antioxidants such as ascorbic acid, methionine, or ethylenediaminetetraacetic acid (EDTA), pro-apoptotic agents such as Bcl-2 inhibitors (e.g., obatoclax, navitoclax, venetoclax, ABT-737, S55746, sabutoclax, or combinations thereof), IAP inhibitors (e.g., AT-406, GDC-0917, LCL-161, GDC-0152, Birinapant, HGS1029, TWX024, AEG35156, or combinations thereof), or MDM2 inhibitors (e.g., Nutlin, ATSP-7041, a smac mimetic, a MCL-1 inhibitor, a Bclxl inhibitor, or a combination thereof). For example, a pharmaceutical composition provided herein can be formulated to include one or more cells designed to express a CAR having the ability to bind to a TRAILshort polypeptide provided herein in combination with one or more pro-apoptotic compounds such as Bcl-2 inhibitors (e.g., Venetoclax, navitoclax, obatoclax ABT-737, S55746, or sabutoclax).

In some cases, when a pharmaceutical composition is formulated to include one or more cells designed to express a CAR having the ability to bind to a TRAILshort polypeptide provided herein, any appropriate concentration of the binder can be used. For example, a pharmaceutical composition provided herein can be formulated to be a liquid that includes from about 1 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 2 mg to about 200 mg, from about 10 mg to about 300 mg, from about 25 mg to about 300 mg, from about 50 mg to about 150 mg, or from about 150 mg to about 300 mg) of a CAR⁺ cell population provided herein per mL. In some cases, when a pharmaceutical composition is formulated to include one or more nucleic acids (e.g., vectors such as viral vectors) encoding at least part of a CAR provided herein, any appropriate concentration of the nucleic acid can be used. For example, a pharmaceutical composition provided herein can be formulated to be a liquid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 2 mg to about 200 mg, from about 10 mg to about 300 mg, from about 25 mg to about 300 mg, from about 50 mg to about 150 mg, or from about 150 mg to about 300 mg) of a nucleic acid provided herein per mL. In another example, a pharmaceutical composition provided herein can be formulated to be a solid or semi-solid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 10 mg to about 300 mg, from about 25 mg to about 300 mg, from about 50 mg to about 150 mg, or from about 150 mg to about 300 mg) of a nucleic acid provided herein.

A pharmaceutical composition provided herein can be in any appropriate form. For example, a pharmaceutical composition provided herein can designed to be a liquid, a semi-solid, or a solid. In some cases, a pharmaceutical composition provided herein can be a liquid solution (e.g., an injectable and/or infusible solution), a dispersion, a suspension, a tablet, a pill, a powder, a microemulsion, a liposome, or a suppository. In some cases, a pharmaceutical composition provided herein can be lyophilized.

This document also provides methods for administering a composition (e.g., a pharmaceutical composition provided herein) containing one or more nucleic acids, vectors, or host cells (e.g., CAR⁺ cells) provided herein) to a mammal (e.g., a human). For example, a composition (e.g., a pharmaceutical composition provided herein) containing one or more nucleic acids, vectors, and/or host cells (e.g., CAR⁺ cells) provided herein can be administered to a mammal (e.g., a human) having a TRAILshort+ cancer to treat that mammal or can be administered to a mammal (e.g., a human) having an infection (e.g., a bacterial or viral infection) to treat that mammal. In some cases, a composition (e.g., a pharmaceutical composition provided herein) containing one or more nucleic acids, vectors, and/or host cells (e.g., CAR⁺ cells) provided herein) can be administered to a mammal (e.g. a human) to reduce the number of cancer cells or infected cells within the mammal and/or to increase the survival of the mammal suffering from cancer.

Any appropriate cancer can be treated using a composition (e.g., a pharmaceutical composition provided herein) containing one or more nucleic acids, vectors, or host cell (e.g., CAR⁺ cells) provided herein. For example, a mammal (e.g., a human) having a TRAILshort⁺ cancer can be treated by administering a composition (e.g., a pharmaceutical composition) provided herein to that mammal. Examples of cancers that can be treated as described herein include, without limitation, TRAILshort⁺ squamous cell carcinoma, lymphoma, cervical cancer, renal cell carcinoma, breast cancer, prostate cancer, ovarian cancer, lung cancer, bladder cancer, head and neck cancer, uterine cancer, esophageal cancer, stomach cancer, colorectal cancer, sarcoma, and pancreatic cancer. For example, a mammal having a TRAILshort+ cancer can be administered a composition (e.g., a pharmaceutical composition) provided herein to treat that mammal (e.g., to reduce the number of cancer cells within the mammal).

When treating an infection as described herein, the infection can be, for example, a chronic infection and/or a viral infection. Examples of infections that can be treated as described herein include, without limitation, HIV, SIV, endogenous retrovirus, anellovirus, circovirus, human herpesvirus, varicella zoster virus, cytomegalovirus, Epstein-Barr virus, polyomavirus, adeno-associated virus, herpes simplex virus, adenovirus, hepatitis B virus, hepatitis C virus, hepatitis D virus, GB virus C, papilloma virus, human T cell leukemia virus, xenotropic murine leukemia virus-related virus, polyomavirus, rubella virus, parvovirus, measles virus, and coxsackie virus infections. In some cases, the infections treated as described herein can be an HIV infection.

Any appropriate method can be used to administer a composition (e.g., a pharmaceutical composition) provided herein to a mammal (e.g., a human). For example, a composition provided herein can be administered to a mammal (e.g., a human), depending on the components of the composition, intravenously (e.g., via an intravenous injection or infusion), subcutaneously (e.g., via a subcutaneous injection), intraperitoneally (e.g., via an intraperitoneal injection), orally, via inhalation, or intramuscularly (e.g., via intramuscular injection). In some cases, the route and/or mode of administration of a composition (e.g., a pharmaceutical composition provided herein) can be adjusted for the mammal being treated.

In some cases, an effective amount of a composition containing one or more nucleic acids, vectors, or host cells (e.g., CAR⁺ cells) provided herein) (e.g., a pharmaceutical composition provided herein) can be an amount that reduces the number of cancer cells or infected cells within a mammal without producing significant toxicity to the mammal. In some cases, an effective amount of a composition containing one or more nucleic acids, vectors, or host cell (e.g., CAR⁺ cells) provided herein (e.g., a pharmaceutical composition provided herein) can be an amount that increases the survival time of a mammal having cancer as compared to a control mammal having comparable cancer and not treated with the composition. In some cases, for example, an effective amount of a composition can contain from about 1×10⁶ to about 1×10¹⁰ CAR T cells (e.g., about 1×10⁶ to about 1×10⁷, about 1×10⁷ to about 1×10⁸, about 1×10⁸ to about 1×10⁹, about 1×10⁹ to about 1×10¹⁰, about 1×10⁷ to about 1×10⁹, or about 1.5×10⁷ to about 1.15×10⁹ CAR T cells). The effective amount can remain constant or can be adjusted as a sliding scale or variable dose depending on the mammal's response to treatment. Various factors can influence the actual effective amount used for a particular application. For example, the severity of cancer when treating a mammal having cancer or the severity of the infection, the route of administration, the age and general health condition of the mammal, excipient usage, the possibility of co-usage with other therapeutic or prophylactic treatments such as use of other agents (e.g., Bcl-2 inhibitors, IAP inhibitors, or MDM2 inhibitors), and the judgment of the treating physician may require an increase or decrease in the actual effective amount of a composition provided herein (e.g., a pharmaceutical composition containing one or more nucleic acids, vectors, or CAR⁺ host cells provided herein) that is administered.

In some cases, an effective frequency of administration of a composition containing one or more nucleic acids, vectors, or host cell (e.g., CAR⁺ cells) provided herein (e.g., a pharmaceutical composition provided herein) can be a frequency that reduces the number of cancer cells within a mammal having cancer without producing significant toxicity to the mammal or reduces the number of infected cells within a mammal without producing significant toxicity. In some cases, an effective frequency of administration of a composition containing one or more nucleic acids, vectors, or host cells (e.g., CAR⁺ cells) provided herein) (e.g., a pharmaceutical composition provided herein) can be a frequency that increases the survival time of a mammal having cancer as compared to a control mammal having comparable cancer and not treated with the composition. Typically, TRAILshort CART cells can be administered once, but in some cases, repeated administration/doses may be used to achieve a deep response. For example, an effective frequency of administration of a pharmaceutical composition provided herein can be from about twice daily to about once a year (e.g., from about twice daily to about once a month, from about twice daily to about once a week, from about once daily to about once a month, or from one once daily to about once a week). In some cases, the frequency of administration of a pharmaceutical composition provided herein can be daily. The frequency of administration of a pharmaceutical composition provided herein can remain constant or can be variable during the duration of treatment. Various factors can influence the actual effective frequency used for a particular application. For example, the severity of the cancer or infection, the route of administration, the age and general health condition of the mammal, excipient usage, the possibility of co-usage with other therapeutic or prophylactic treatments such as use of other agents (e.g., Bcl-2 inhibitors, IAP inhibitors, or MIDM2 inhibitors), and the judgment of the treating physician may require an increase or decrease in the actual effective frequency of administration of a composition provided herein

In some cases, an effective duration of administration of a composition containing one or more nucleic acids, vectors, or host cells (e.g., CAR⁺ cells) provided herein (e.g., a pharmaceutical composition provided herein) can be a duration that reduces the number of cancer cells or infected cells within a mammal without producing significant toxicity to the mammal. In some cases, an effective duration of administration of a composition containing one or more nucleic acids, vectors, or host cells (e.g., CAR⁺ cells) provided herein) (e.g., a pharmaceutical composition provided herein) can be a duration that increases the survival time of a mammal having cancer as compared to a control mammal having comparable cancer and not treated with the composition. For example, an effective duration of administration of a pharmaceutical composition provided herein can vary from a single time point of administration to several weeks to several months (e.g., 4 to 12 weeks). Multiple factors can influence the actual effective duration used for a particular application. For example, the severity of the cancer or infection, the route of administration, the age and general health condition of the mammal, excipient usage, the possibility of co-usage with other therapeutic or prophylactic treatments such as use of other agents (e.g., Bcl-2 inhibitors, IAP inhibitors, or MDM2 inhibitors), and the judgment of the treating physician may require an increase or decrease in the actual effective duration of administration of a composition provided herein.

The invention will be further described in the following examples, which do not limit the scope of the invention described in the claims.

### EXAMPLES

### Example 1: Humanization of Anti-TRAILshort Antibodies

The generation of TRAILshort antibodies that are specific to the 11 C terminal amino acids unique to TRAILshort (SEQ ID NO:35; FIG. 1) is described, for example, in Schnepple et al., 2011 J Biol Chem 286:35742-35754. Murine monoclonal antibody TRAILs 2.2 was humanized to create antibody Ab866. See, e.g., U.S. Patent Application Publication No. 20190367626. FIG. 3A contains the amino acid sequences of murine and humanized anti-TRAIL short heavy chains.

### Example 2—TRAILshort CARs (TsCARs) from Binders Having the Ability to Bind to a Human TRAILshort Polypeptide

TRAILshort CARs were made using the following combinations:
(1) TRAILshort scFv (L2H)-CD8 hinge and TM domain-41BB-CD3z signaling domain
(2) TRAILshort scFv (H2L)-CD8 hinge and TM domain-41BB-CD3z signaling domain
(3) TRAILshort scFv (L2H)-CD28 hinge and TM domain-CD28-CD3z signaling domain
(4) TRAILshort scFv (H2L)-CD28 hinge and TM domain-CD28-CD3z. signaling domain

FIG. 4 shows the nucleic acid sequences encoding ScFv with HC3 and LC2, as well as codon optimized nucleic acid sequences encoding HC3 and LC2. FIG. 5 shows nucleic acid sequences encoding ScFv with LC2 and HC2, as well as codon optimized nucleic acid sequences encoding HC3 and LC2. A schematic of the TRAILshort CAR used in the following examples is shown in FIG. 7B.

### Example 3—TsCAR T Cells do not have any T Cell Toxicity

An experiment was designed to determine whether TsCAR T cells target CD4 T cells, CD8 T cells, or total T cells, as some subsets of T cells are known to express TRAIL. See Table 3 for the details regarding the experimental design. The following types of cells were used in the experiment:

UTD: Untransduced T cells in which the cells go through the same treatment as TsCAR T cells but they are not transduced with viral particles that express the CAR.

K161: These are TsCAR T cells expressing the TsCAR shown in FIG. 7B. The CAR constructs were designed and cloned into a lentiviral expression vector. Expression vectors were cotransfected with packaging and enveloping plasmids into 293T cells. Viral particles were concentrated in the media of the transfected 293T cells.

Target: T cells from the same donor cultured alone.

M UTD/M K161: These are the TsCAR T cells cultured alone to serve as negative controls (no proliferation is expected).

PI UTD/PI K161: These are the TsCAR T cells cultured with PMA/ionomycin (chemicals that activate T cells) to serve as positive controls (strong activation is expected).

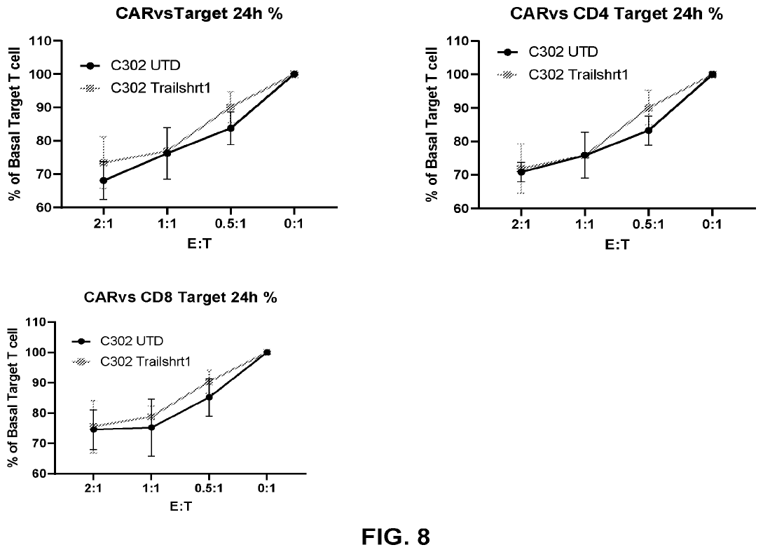

In this experiment, K161 cells or UTD cells were co-cultured with T cells from the same donor (target cells) at the ratios indicated in Table 3. The T cells were pre-stained with CFSE dye. The number of TsCAR T cells, total T cells, CD4⁺ cells, and CD8⁺ cells was monitored for three days, with the number of cells determined using flow cytometry and counting beads. As shown in FIG. 8, the TsCAR T cells do not target total T cells (upper left panel in FIG. 8), CD4⁺ cells (upper right panel in FIG. 8), or CD8⁺ T cells (bottom left panel in FIG. 8) as the number of target cells would have been significantly lower in Ts CAR T cell-T cell co-cultures if the TsCAR T cells targeted any of the T cell populations. The results in FIG. 8 were after 24 hours. Similar results were observed after 48 and 72 hours.

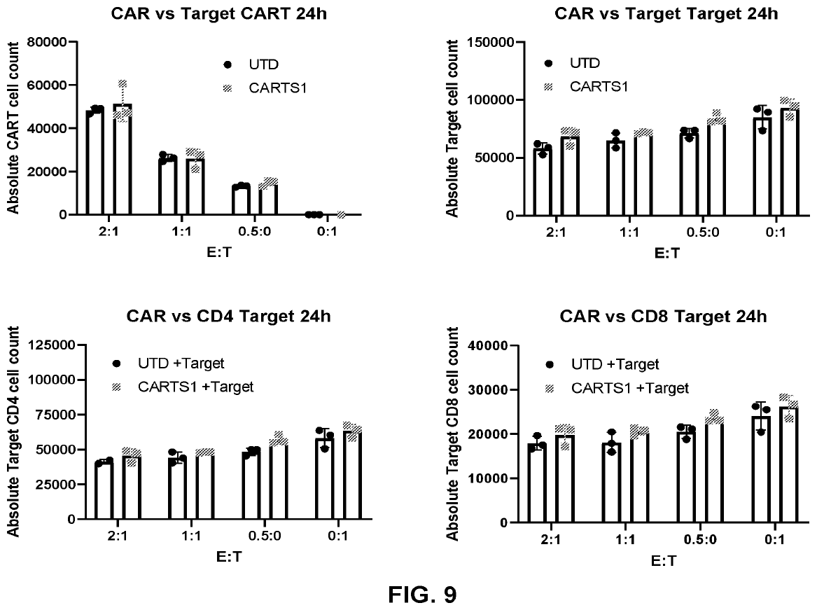

The experiment was repeated, and the CAR T/UTD cells, total T cells, CD4⁺ T cells, and CD8⁺ T cells were counted using counting beads with flow cytometry. If TsCAR T cells were actively targeting any of the target cells, an increased number of TsCAR T cells would be expected compared to UTD cells and as a consequence of TsCAR T cell targeting, it would be expected to see a decreased number of target cells in TsCAR T cell-target T cell co-cultures. As shown in FIG. 9, however, these results were not observed. Accordingly, this experiment also demonstrated that TsCAR T cells do not target T cells. The results in FIG. 9 were after 24 hours. Similar results were observed after 48 and 72 hours.

### Example 4—Cytotoxicity of TsCAR T Cells Against Various Cell Lines

Target cells (cells listed as cell lines in Table 4) were generated that expressed a luciferase gene. The target cells were co-cultured with either TsCAR T cells or UTD cells at varying E:T ratio (E: Effector cells=UTD or TsCAR T cells; and T: Target cells=tumor cell lines). Luciferin (luciferase target) was added to each well and the signal generated by luciferase was measured. Target cells cultured by themselves were considered as 100% and the signal differences between each well was calculated as % killing. In the case of TsCAR T cell specific tumor cell killing, the signal from TsCAR T cell-tumor cell line co-cultures would be lower than UTD therefore percent killing would be higher in TsCAR T cell-tumor cell cocultures.

ARG77, BCWM, Je-Kol, and Jurkat cell lines were co-cultured with either UTD or TsCAR T cells with varying E:T ratios (shown in FIG. 10) in a cytotoxicity assay. There was a TsCAR specific killing in these cells as the percent killing was higher in TsCAR T cell co-culture wells compared to the UTD co-cultures. See FIG. 10.

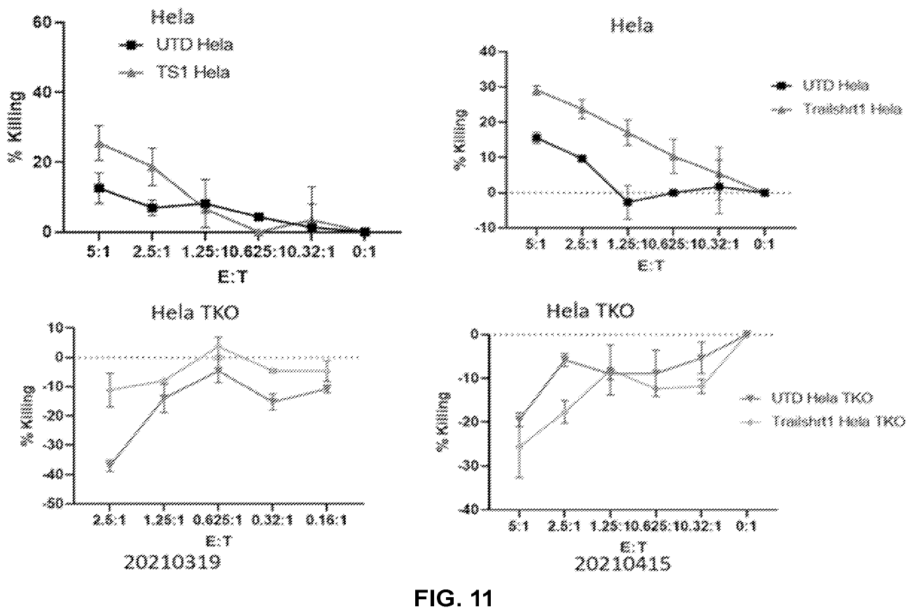

To test if the TsCAR T cell killing was specific to the TRAILshort isoform, wild type HeLa and HeLa cells with a TRAIL knockout (KO) were used. As shown in FIG. 11, TsCAR T cells were cytotoxic against wild type HeLa cells, which express TRAILshort, especially at higher E:T ratios. However, HeLa KO cells were not targeted by TsCAR T cells, demonstrating the specificity of the TsCAR T cells. The TsCAR T cells also were not effective against HBL1, RPM18226, MCF7, BXPC3, or L3.6 cell lines.

TABLE 3
TsCAR T cell activity against CD4⁺ and CD8⁺ T cells- Experimental design

| | 2:1 | | | 1:1 | | | 0.5:1 | | | 0:1 | | |
|---|---|---|---|---|---|---|---|---|---|---|---|---|
| | 1 | 2 | 3 | 4 | 5 | 6 | 7 | 8 | 9 | 10 | 11 | 12 |
| A | UTD | UTD | UTD | UTD | UTD | UTD | UTD | UTD | UTD | Target | Target | Target |
| B | K161 | K161 | K161 | K161 | K161 | K161 | K161 | K161 | K161 | Target | Target | Target |
| C | M UTD | M UTD | M UTD | PI UTD | PI UTD | PI UTD | | | | | | |
| D | M K161 | M K161 | M K161 | PI K161 | PI K161 | PI K161 | | | | | | |

TABLE 4
Summary of Cytotoxicity Experiments

| Cell line | Cell type | Disease | 1* | 2 | 3 | 4 | 5 |
|---|---|---|---|---|---|---|---|
| ARH77 | Suspension | Plasma cell leukemia | | | | | Positive |
| BCWM | Suspension | Waldenstrom macroglobulinemia | | | | | Positive |
| Jurkat | Suspension | acute T cell leukemia | Positive | Positive | | | |
| Jurkat PMA | Suspension | acute T cell leukemia | Positive | Positive | | | |
| JeKo-1 | Suspension | Lymphoma | | Positive | Positive | | Positive |
| HeLa WT | Adherent | Adenocarcinoma | | | Negative | Positive | Positive |
| HeLa TRAIL KO | Adherent | Adenocarcinoma (no TRAIL) | | | Negative | Negative | Negative |
| Jurkat I14 | Suspension | Acute T cell leukemia | Negative | Negative | | | |
| Jurkat I14 PMA | Suspension | Acute T cell leukemia | Negative | Negative | | | |
| Jurkat C9M | Suspension | Acute T cell leukemia | Discarded | | | | |
| Jurkat B20 | Suspension | Acute T cell leukemia | Discarded | | | | |
| HBL-1 | Suspension | Diffuse large B-cell lymphoma | | | Negative | | |
| RPMI8226 | Suspension | Multiple myeloma | | | Negative | | |
| BxPC3 | Adherent | Pancreatic cancer | | | Negative | Negative | |
| L3.6 | Adherent | Pancreatic cancer | Negative | Negative | | Negative | |
| MCF7 old | Adherent | Breast cancer cells | Negative | | | | |
| MCF7 new | Adherent | Breast cancer cells | | | | Negative | |

*1-5 represent different experiments.

### Example 5—Dual TsCAR/CAR19 T Cell Efficacy

In this experiment, T cells were transduced with viral particles expressing both the CD19CAR and TsCAR. FIG. 7B contains a schematic of both CARs. As controls, UTD, TsCAR T cells, and CD19 CAR T cells were also generated. The cancer cell lines (ARH77, BCWM, HeLa, HeLa TKO, or JeKo-1) were co-cultured with UTD, TsCAR T cells, CD19 CAR T cells, and Dual CAR T cells in a cytotoxicity assay. While all of the cell lines tested were TRAIL positive, they behaved differently when cocultured with the CAR T cells. As shown in FIG. 12, T cells expressing both CARs were cytotoxic to ARH88 and JeKo-1 cell lines in vitro. Dual CAR T cells were as effective as CD19 CAR T cells in the ARH77 and JeKo-1 cell lines.

A proliferation assay was performed by co-culturing target cancer cell lines (HeLa, HeLa TKO, ARH77, BCWM, or JeKo) with either UTD or TsCAR T cells with 1:1 E:T ratio, or Dual CAR T cells. UTD and TsCAR T cells were cultured alone (media-negative control) where expected not to proliferate. PI (PMA/Inomycine) activates T cells and served as a positive control. Cancer cell lines and CAR T cells were co-cultured for 5 days and the number of CAR T or UTD cells were determined by using flow cytometry with counting beads. CAR T cell activity was shown to correlate with CAR T cell number. Dual CAR T cell proliferation was comparable to the CD19 CAR T cells showing effective proliferative activity of Dual CAR T cells. See, FIG. 13.

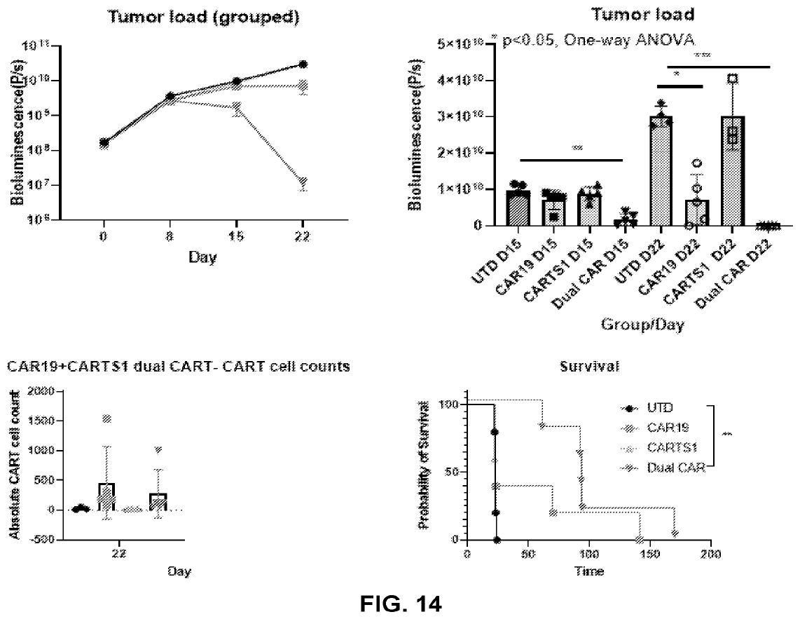

Four groups of NSG mice (n=5 mice each) were generated. In each mouse, 1M Luciferase⁺JeKo-1 cell line were transplanted and then two weeks later, each group of mice were transplanted with either 1M UTD, CD19 CAR T cells, TsCAR T cells, or Dual CAR T cells. The tumor load was monitored regularly by injecting luciferin into the mice and measuring the luciferase signal intensity. As shown in FIG. 14, the tumor load was lower in Dual CAR T cell transplanted mice compared to the other groups. CAR T cell counts were performed by bleeding the mice from tail. In summary, the red blood cells were lysed from the blood that was collected and the T cells were counted by using flow cytometry. The survival curve also is shown in FIG. 14.

### Example 6—Combining TsCAR T Cells with Bcl-2 Inhibitors

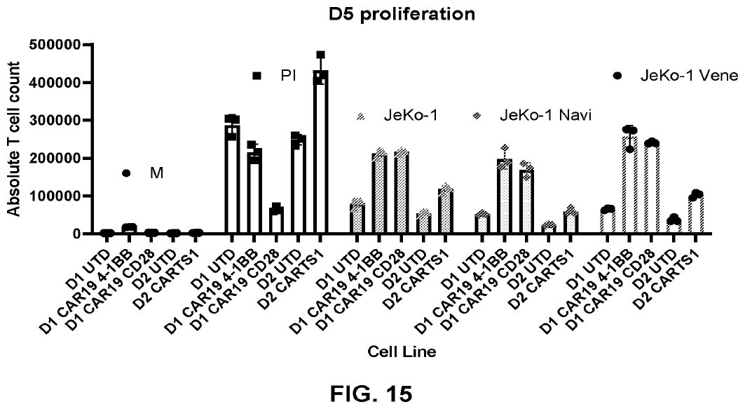

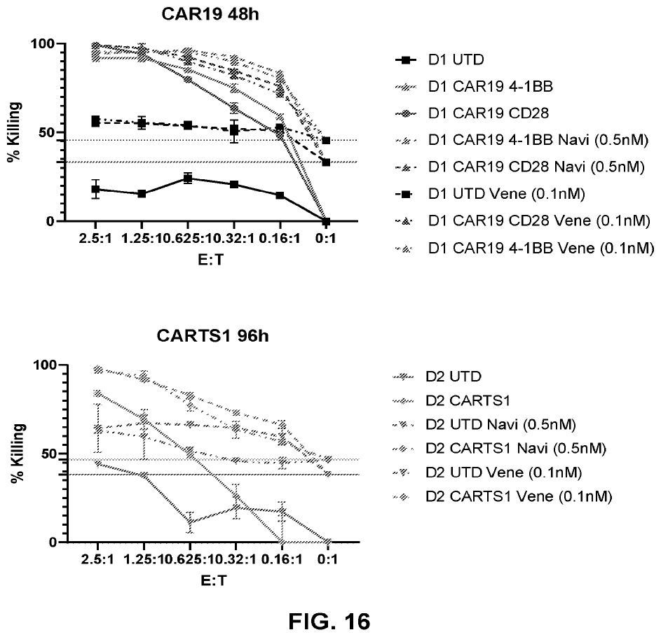

TsCAR T cells combined with Navitoclax (BCL-2 inhibitor) treatment resulted in decreased CAR T cell proliferation against the JeKo-1 cell line while the combination with Venetoclax (BCL-2 inhibitor) did not lead to decreased proliferation. See, FIG. 15. In FIG. 15, D1 refers to donor 1 and D2 refers to Donor 2. In the results shown in FIG. 15, UTD cells, CD19 CAR T cells (two different CD19 CARS-CAR19 4-1BB and CAR19 CD28, which express FDA-approved CAR19 constructs that are used in the clinic to treat ALL (CAR19 4-1BB) and DLBCL (both CARS); the main difference between these is their 4-1BB or CD28 co-stimulatory domains), or TsCAR T cells, were cultured as follows: (i) alone (M in FIG. 15) as a negative control with no proliferation expected, (ii) in the presence of PI (PMA/Ionomycin) as a positive control where proliferation is expected; (iii) co-cultured with JeKo-1 cell line (JeKo-1 in FIG. 15); (iv) co-cultured with JeKo-1 cells in the presence of Navitoclax (JeKo-1 Navi in FIG. 15); or (v) co-cultured with JeKo-1 cell line in the presence of Venetoclax (JeKo-1 Vene in FIG. 15). Overall, the number of TsCAR T cells was decreased in JeKo-1 Navi compared to the JeKo-1 but the number of TsCAR T cells in JeKo-1 Vene was not decreased compared to JeKo-1 TsCAR T cells showing the safety of combining CAR T cells and venetoclax. As shown in FIG. 16, both venetoclax and navitoclax combination therapies with TsCAR T cells resulted in increased cytotoxicity when these two drugs were added to the TsCAR T cell-JeKo-1 co-culture. As there was increased cytotoxicity with TsCAR T cells along with CAR19 CART cells, the results were not specific to Ts CART cells.

TsCAR T cells combined with Navitoclax treatment increased TsCAR T cell cytotoxicity when co-cultured with JeKo-1, but not with Jurkat, MCF7, ARH77, Hela or HeLa Trail KO cell lines.

### Example 7—TsCAR T Cells Reduce JeKo-1 Cell Proliferation in a Mouse Xenograft Model

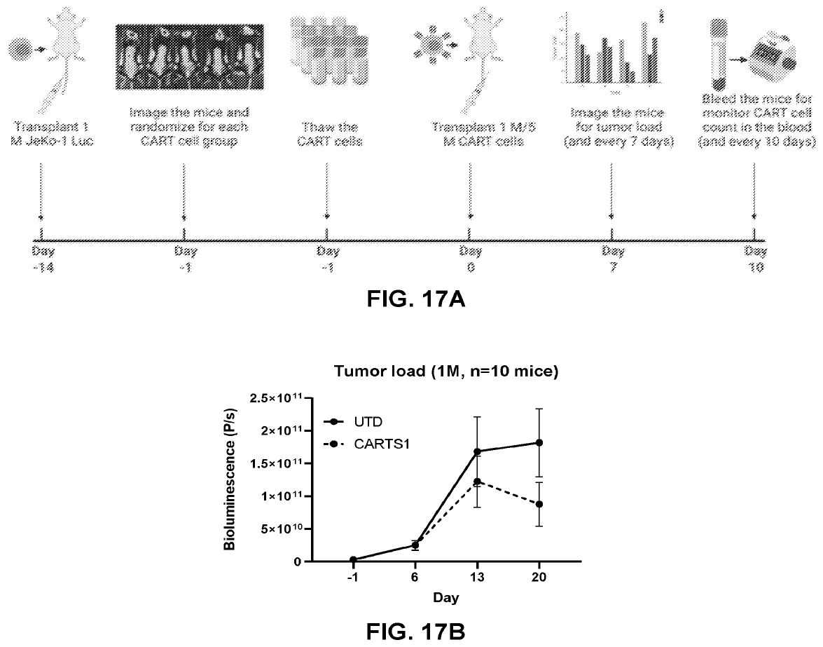

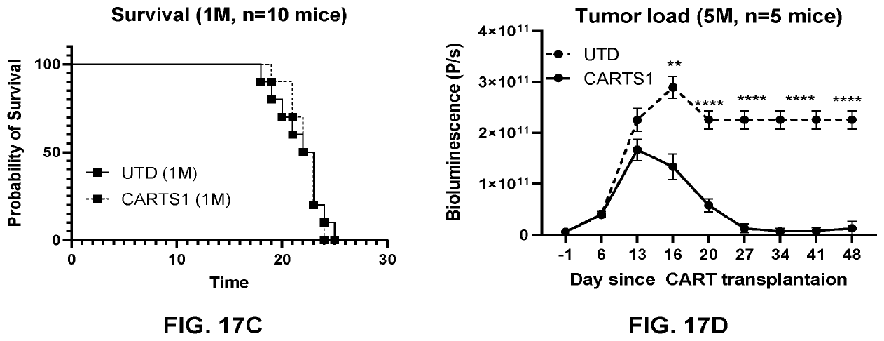

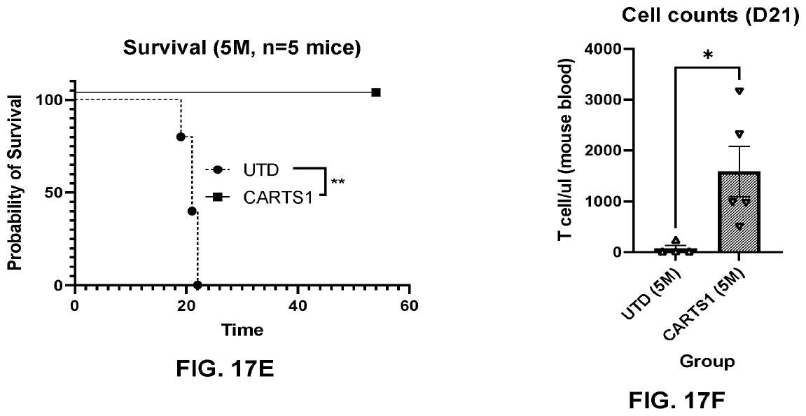

NSG mice were engrafted with the luciferase⁺ mantle cell lymphoma JeKo-1 cells (1×10⁶ or 5×10⁶ cells i.v.; FIG. 17A). Two weeks after engraftment, mice underwent bioluminescence imaging to confirm engraftment, and then were randomized to receive TRAILshort CART cells (1×10⁶ or 5×10⁶ cells i.v.) or control UTD (1×10⁶ or 5×10⁶ cells i.v.). Mice underwent serial bioluminescent imaging and also were monitored for survival. Bioluminescence after luciferin injection to the mice transplanted with 1M UTD or CARTS1 cells is plotted in FIG. 17B according to time (n=10 mice/group). Survival is shown in FIG. 17C; all of the mice died between days 18 and 25. Bioluminescence of the UTD and CARTS1 mice transplanted with 5M UTD or CARTS1 cells is plotted in FIG. 17D. The CARTS1 mice had significantly less tumor burden starting on day 16. Survival is shown in FIG. 17E; the CARTS1 (5M) mice showed increased survival compared to the UTD (5M) mice. The CARTS1 mice still had low tumor burden when they were sacrificed at day 55 due to GVHD, which was an expected end point since human T cell derived CART cells had been transplanted into the mice. CART cell numbers in peripheral blood samples collected from the mice at day 21 are plotted in FIG. 17F. The CARTS1 (5M) mice had significantly higher numbers of CART cells than the UTD controls. No T cells were detected in UTD (1M), CARTS (1M), or UTD (5M) mice that survived up to 21 days (not shown). Taken together, these in vivo studies demonstrated that treatment with TRAILshort CART cells resulted in complete remission and prolonged survival of JeKo-1 xenografts, and increased T cell proliferation in the peripheral blood 21 days after CART treatment.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A chimeric antigen receptor comprising an antigen binding domain, a hinge, a transmembrane domain, and one or more signaling domains, wherein said antigen binding domain comprises:
a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one amino acid addition, deletion, or substitution), and
a light chain variable domain or region comprising the amino acid sequence set forth in SEQ ID NO:9 (or SEQ ID NO:9 with one, two, or three amino acid additions, deletions, or substitutions), the amino acid sequence Gly-Ala-Ser (GAS) (or amino acid sequence GAS with one amino acid addition, deletion, or substitution), and SEQ ID NO:10 (or SEQ ID NO:10 with one, two, or three amino acid additions, deletions, or substitutions).

2. The chimeric antigen receptor of claim 1, wherein said antigen binding domain comprises the ability to bind to a human TRAILshort polypeptide.

3-11. (canceled)

12. The chimeric antigen receptor of claim 1, wherein said one or more signaling domains comprises one or more of a 4-1BB intracellular signaling domain, a CD28 intracellular signaling domain, or a CD3ζ intracellular signaling domain.

13-14. (canceled)

15. An isolated population of cells, wherein at least one cell of said population comprises a nucleic acid encoding a chimeric antigen receptor comprising an antigen binding domain, a hinge, a transmembrane domain, and one or more signaling domains, wherein said antigen binding domain comprises:
a heavy chain variable domain or region comprising the amino acid sequences set forth in SEQ ID NO:1 (or SEQ ID NO: 1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one amino acid addition, deletion, or substitution), and
a light chain variable domain or region comprising the amino acid sequence set forth in SEQ ID NO:9 (or SEQ ID NO:9 with one, two, or three amino acid additions, deletions, or substitutions), the amino acid sequence Gly-Ala-Ser (GAS) (or amino acid sequence GAS with one amino acid addition, deletion, or substitution), and SEQ ID NO: 10 (or SEQ ID NO:10 with one, two, or three amino acid additions, deletions, or substitutions).

16. The population of claim 15, wherein said at least one cell expresses said nucleic acid and comprises said chimeric antigen receptor on the cell surface.

17. The population of claim 15, wherein said at least one cell comprises nucleic acid encoding a second chimeric antigen receptor.

18. (canceled)

19. The population of claim 17, wherein said at least one cell expresses said nucleic acid encoding said second chimeric antigen receptor and comprises said second chimeric antigen receptor on the cell surface.

20-22. (canceled)

23. The population of claim 15, wherein said cell is a T cell, a stem cell, or an NK cell.

24. An isolated nucleic acid comprising a nucleic acid sequence encoding a chimeric antigen receptor comprising an antigen binding domain, a hinge, a transmembrane domain, and one or more signaling domains, wherein said antigen binding domain comprises:
a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one amino acid addition, deletion, or substitution), and
a light chain variable domain or region comprising the amino acid sequence set forth in SEQ ID NO:9 (or SEQ ID NO:9 with one, two, or three amino acid additions, deletions, or substitutions), the amino acid sequence Gly-Ala-Ser (GAS) (or amino acid sequence GAS with one amino acid addition, deletion, or substitution), and SEQ ID NO:10 (or SEQ ID NO:10 with one, two, or three amino acid additions, deletions, or substitutions).

25-26. (canceled)

27. A method of making a chimeric antigen receptor positive (CAR⁺) cell, wherein said method comprises introducing a nucleic acid encoding said CAR into a cell, wherein said cell expresses said CAR, thereby making said CAR⁺ cell, and wherein said CAR comprises an antigen binding domain, a hinge, a transmembrane domain, and one or more signaling domains, wherein said antigen binding domain comprises:
a heavy chain variable domain or region comprising the amino acid sequences set forth in SEQ ID NO:1 (or SEQ ID NO: 1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one amino acid addition, deletion, or substitution), and
a light chain variable domain or region comprising the amino acid sequence set forth in SEQ ID NO:9 (or SEQ ID NO:9 with one, two, or three amino acid additions, deletions, or substitutions), the amino acid sequence Gly-Ala-Ser (GAS) (or amino acid sequence GAS with one amino acid addition, deletion, or substitution), and SEQ ID NO:10 (or SEQ ID NO:10 with one, two, or three amino acid additions, deletions, or substitutions).

28-45. (canceled)

46. A method of treating a mammal having cancer or an infection, wherein said method comprises administering, to said mammal, a composition comprising a population of cells, wherein at least one cell of said population comprises a nucleic acid encoding a chimeric antigen receptor comprising an antigen binding domain, a hinge, a transmembrane domain, and one or more signaling domains, wherein said antigen binding domain comprises:
a heavy chain variable domain or region comprising the amino acid sequences set forth in SEQ ID NO: 1 (or SEQ ID NO: 1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one amino acid addition, deletion, or substitution), and
a light chain variable domain or region comprising the amino acid sequence set forth in SEQ ID NO:9 (or SEQ ID NO:9 with one, two, or three amino acid additions, deletions, or substitutions), the amino acid sequence Gly-Ala-Ser (GAS) (or amino acid sequence GAS with one amino acid addition, deletion, or substitution), and SEQ ID NO:10 (or SEQ ID NO:10 with one, two, or three amino acid additions, deletions, or substitutions).

47. The method of claim 46, wherein said mammal is a human.

48. The method of claim 46, wherein said mammal has said cancer, and wherein said cancer is a TRAILshort⁺ cancer.

49. The method of claim 48, wherein said TRAILshort⁺ cancer is a TRAILshort⁺ squamous cell carcinoma, lymphoma, cervical cancer, renal cell carcinoma, breast cancer, prostate cancer, ovarian cancer, lung cancer, bladder cancer, head and neck cancer, uterine cancer, esophageal cancer, stomach cancer, colorectal cancer, sarcoma, or pancreatic cancer.

50. The method of claim 48, wherein the number of cancer cells within said mammal is reduced following said administering step.

51-52. (canceled)

53. The method of claim 48, wherein said method comprises administering, to said mammal, a pro-apoptotic compound.

54. The method of claim 53, wherein said pro-apoptotic compound is a Bcl-2 inhibitor, an IAP inhibitor, or a MDM2 inhibitor.

55. The method of claim 46, wherein said mammal comprises said infection.

56. The method of claim 55, wherein said infection is a chronic infection.

57. The method of claim 55, wherein said infection is selected from the group consisting of human immunodeficiency virus (HIV) infection, hepatitis B virus (HBV) infection, hepatitis C virus (HCV) infection, human papilloma virus (HPV) infection, tuberculosis (TB) infection, cytomegalovirus (CMV) infection, and Epstein-Barr virus (EBV) infection.

58-65. (canceled)


SEQUENCE LISTING (159 sequences)

SEQ ID NO: 1 - protein, 8 aa, synthetic construct
GYIFTNND

SEQ ID NO: 2 - protein, 8 aa, synthetic construct
IDPGDGRT

SEQ ID NO: 3 - protein, 11 aa, synthetic construct
GRGGYEFGIDY

SEQ ID NO: 4 - protein, 25 aa, synthetic construct
QVQLVQSGAEVKKPGSSVKVSCKSS

SEQ ID NO: 5 - protein, 17 aa, synthetic construct
MNWVRQAPGQGLDWMGG

SEQ ID NO: 6 - protein, 38 aa, synthetic construct
KYNEKFKGRVTISADIFSNTAYMELNSLTSEDTAVYFC

SEQ ID NO: 7 - protein, 11 aa, synthetic construct
WGQGTTVTVSS

SEQ ID NO: 8 - protein, 118 aa, synthetic construct
QVQLVQSGAEVKKPGSSVKVSCKSSGYIFTNNDMNWVRQAPGQGLDWMGGIDPGDGRTKYNEKFKGRVTISADIFSNTAYMELNSLTSEDTAVYFCGRGGYEFGIDYWGQGTTVTVSS

SEQ ID NO: 9 - protein, 12 aa, synthetic construct
QSLLNSGNQKNS

SEQ ID NO: 10 - protein, 9 aa, synthetic construct
QNDHSFPLT

SEQ ID NO: 11 - protein, 26 aa, synthetic construct
DIVMTQSPDSLAVSLGERATINCKSS

SEQ ID NO: 12 - protein, 17 aa, synthetic construct
LAWYQQKPGQPPKLLIY

SEQ ID NO: 13 - protein, 36 aa, synthetic construct
TRESGVPDRFSGSGSGTDFTLTISSLQAEDVAVYYC

SEQ ID NO: 14 - protein, 10 aa, synthetic construct
FGGGTKLEIK

SEQ ID NO: 15 - protein, 113 aa, synthetic construct
DIVMTQSPDSLAVSLGERATINCKSSQSLLNSGNQKNSLAWYQQKPGQPPKLLIYGASTRESGVPDRFSGSGSGTDFTLTISSLQAEDVAVYYCQNDHSFPLTFGGGTKLEIK

SEQ ID NO: 16 - protein, 118 aa, Mus musculus
QVQLQQSGPELVKPGASVKISCKASGYIFTNNDMNWVKQRPGQGLEWIGGIDPGDGRTKYNEKFKGKATLTADKFSNTVYMQLSSLTSENSAVYFCGRGGYEFGIDYWGQGTSVTVSS

SEQ ID NO: 17 - protein, 464 aa, Mus musculus
MGWTLVFLFLLSVTAGVHSQVQLQQSGPELVKPGASVKISCKASGYIFTNNDMNWVKQRPGQGLEWIGGIDPGDGRTKYNEKFKGKATLTADKFSNTVYMQLSSLTSENSAVYFCGRGGYEFGIDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 118 aa, synthetic construct
QVQLVQSGAEVKKPGATVKISCKVSGYIFTNNDMNWVQQAPGKGLEWMGGIDPGDGRTKYNEKFKGRVTITADESTSTAYMELSSLRSEDTAVYYCGRGGYEFGIDYWGQGTLVTVSS

SEQ ID NO: 19 - protein, 464 aa, synthetic construct
MGWTLVFLFLLSVTAGVHSQVQLVQSGAEVKKPGATVKISCKVSGYIFTNNDMNWVQQAPGKGLEWMGGIDPGDGRTKYNEKFKGRVTITADESTSTAYMELSSLRSEDTAVYYCGRGGYEFGIDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 118 aa, synthetic construct
QVQLVQSGAEVKKPGASVKVSCKASGYIFTNNDMNWVRQAPGQGLEWMGGIDPGDGRTKYNEKFKGRVTMTRDTSTNTVYMELSSLTSEDTAVYFCGRGGYEFGIDYWGQGTTVTVSS

SEQ ID NO: 21 - protein, 464 aa, synthetic construct
MGWTLVFLFLLSVTAGVHSQVQLVQSGAEVKKPGASVKVSCKASGYIFTNNDMNWVRQAPGQGLEWMGGIDPGDGRTKYNEKFKGRVTMTRDTSTNTVYMELSSLTSEDTAVYFCGRGGYEFGIDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 464 aa, synthetic construct
MGWTLVFLFLLSVTAGVHSQVQLVQSGAEVKKPGSSVKVSCKSSGYIFTNNDMNWVRQAPGQGLDWMGGIDPGDGRTKYNEKFKGRVTISADIFSNTAYMELNSLTSEDTAVYFCGRGGYEFGIDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 118 aa, synthetic construct
QVQLVESGAEVKKPGASVKVSCKVSGYIFTNNDMNWVRQAPGEGLEWMGGIDPGDGRTKYNEKFKGRVTMTEDTSTDTAYMELSSLRSEDTAVYYCGRGGYEFGIDYWGQGTTVTVSS

SEQ ID NO: 24 - protein, 464 aa, synthetic construct
MGWTLVFLFLLSVTAGVHSQVQLVESGAEVKKPGASVKVSCKVSGYIFTNNDMNWVRQAPGEGLEWMGGIDPGDGRTKYNEKFKGRVTMTEDTSTDTAYMELSSLRSEDTAVYYCGRGGYEFGIDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 113 aa, Mus musculus
DIVMTQSPSSLSVSAGEKVTMSCKSSQSLLNSGNQKNSLAWYQQKPGRPPTLLISGASTRESGVPDRFTGSGSGTDFTLTISSVQAEDLAVYYCQNDHSFPLTFGAGTKLELK

SEQ ID NO: 26 - protein, 240 aa, Mus musculus
MVSSAQFLGLLLLCFQGTRCDIVMTQSPSSLSVSAGEKVTMSCKSSQSLLNSGNQKNSLAWYQQKPGRPPTLLISGASTRESGVPDRFTGSGSGTDFTLTISSVQAEDLAVYYCQNDHSFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 113 aa, synthetic construct
DVVMTQSPDSLAVSLGERATINCKSSQSLLNSGNQKNSLAWYQQKPGQPPKLLIYGASTRESGVPDRFSGSGSGTDFTLTISSLQAEDVAVYYCQNDHSFPLTFGQGTKLEIK

SEQ ID NO: 28 - protein, 240 aa, synthetic construct
MVSSAQFLGLLLLCFQGTRCDVVMTQSPDSLAVSLGERATINCKSSQSLLNSGNQKNSLAWYQQKPGQPPKLLIYGASTRESGVPDRFSGSGSGTDFTLTISSLQAEDVAVYYCQNDHSFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 240 aa, synthetic construct
MVSSAQFLGLLLLCFQGTRCDIVMTQSPDSLAVSLGERATINCKSSQSLLNSGNQKNSLAWYQQKPGQPPKLLIYGASTRESGVPDRFSGSGSGTDFTLTISSLQAEDVAVYYCQNDHSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 113 aa, synthetic construct
DIVMTQSPDSLAVSLGERATINCKSSQSLLNSGNQKNSLAWYQQRPGHPPKLLLYGASTRESGVPDRFSGSGSGTDFTLTISSLQAEDVAVYYCQNDHSFPLTFGGGTKVEIK

SEQ ID NO: 31 - protein, 240 aa, synthetic construct
MVSSAQFLGLLLLCFQGTRCDIVMTQSPDSLAVSLGERATINCKSSQSLLNSGNQKNSLAWYQQRPGHPPKLLLYGASTRESGVPDRFSGSGSGTDFTLTISSLQAEDVAVYYCQNDHSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 113 aa, synthetic construct
EIVLTQSPDSLAVSLGERATINCKSSQSLLNSGNQKNSLAWYQHKPGRPPKLLIYGASTRESGVPDRFSGSGSGEDFTLTISSLQAEDVAVYYCQNDHSFPLTFGPGTKVDLK

SEQ ID NO: 33 - protein, 240 aa, synthetic construct
MVSSAQFLGLLLLCFQGTRCEIVLTQSPDSLAVSLGERATINCKSSQSLLNSGNQKNSLAWYQHKPGRPPKLLIYGASTRESGVPDRFSGSGSGEDFTLTISSLQAEDVAVYYCQNDHSFPLTFGPGTKVDLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 101 aa, Homo sapiens
MAMMEVQGGPSLGQTCVLIVIFTVLLQSLCVAVTYVYFTNELKQMQDKYSKSGIACFLKEDDSYWDPNDEESMNSPCWQVKWQLRQLVRKTPRMKRLWAAK

SEQ ID NO: 35 - protein, 11 aa, Homo sapiens
TPRMKRLWAAK

SEQ ID NO: 36 - protein, 242 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 37 - DNA, 726 bp, synthetic construct
  source 1..726: mol_type = other DNA
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctca

SEQ ID NO: 38 - DNA, 354 bp, synthetic construct
  source 1..354: mol_type = other DNA
caagtgcagttggtgcagagcggagccgaggtcaagaagccgggctcatcagtgaaagtgtcctgcaagagctccggatacatcttcactaacaacgacatgaactgggtcagacaggcgcccggccagggtctggactggatggggggaatcgaccctggagatggcagaaccaagtacaacgaaaagttcaagggacgcgtgaccatctctgcggatatcttcagcaacaccgcttacatggagctgaacagcctgacgtccgaggacaccgctgtctacttttgcggccggggtggatacgaattcggaattgattactggggccaggggacgaccgtgaccgtcagcagc

SEQ ID NO: 39 - DNA, 354 bp, synthetic construct
  source 1..354: mol_type = other DNA
caagtgcagctcgtgcaatccggcgcagaggttaaaaaaccaggaagctcagtcaaggtcagttgtaagagctctggttacatatttacgaataatgacatgaattgggtaagacaggctcctggacagggtctggattggatgggtgggatagaccctggagacgggcggacgaaatacaacgagaagtttaaagggagagtgacgatcagcgcggacatattctcaaatactgcgtatatggaactgaactccctgactagcgaggacacagcggtgtatttctgtggtcggggcgggtatgaatttgggattgattactggggccaaggtacgaccgtgaccgtttccagc

SEQ ID NO: 40 - DNA, 339 bp, synthetic construct
  source 1..339: mol_type = other DNA
gatatcgtgatgactcagtccccggattccctggccgtgtccctgggcgaacgggccaccatcaactgcaagagctcacagtcccttctgaactccggcaaccagaagaactccctcgcgtggtatcagcagaaaccggggcagccaccgaagttgctgatctacggtgcctccactcgggaatccggagtgccggataggttctcgggctccgggtccggcacagacttcaccctcaccatttcatcgctgcaagcggaggacgtggccgtgtactactgccaaaacgaccacagcttccctctcactttcggaggcggtactaagctggagatcaag

SEQ ID NO: 41 - DNA, 339 bp, synthetic construct
  source 1..339: mol_type = other DNA
gatattgtaatgacgcaatcaccagattcactggcagtgtctctcggcgaaagggctacaattaattgtaagagctcacaaagtttgctgaactctgggaaccaaaaaaactctctggcttggtaccaacagaagcctgggcaacctcccaaattgttgatttacggagcatccacgagagagagtggcgtcccagatcgattttcagggagcggaagcggcaccgattttactctcactatcagttctttgcaggctgaagacgtggctgtctattattgccagaacgaccactctttcccacttacctttggtggggggactaagctcgaaataaag

SEQ ID NO: 42 - DNA, 354 bp, synthetic construct
  source 1..354: mol_type = other DNA
caagtgcagttggtgcagagcggagccgaggtcaagaagccgggcgcatcagtgaaagtgtcctgcaaggcgtccggatacatcttcactaacaacgacatgaactgggtcagacaggcgcccggccagggtctggagtggatggggggaatcgaccctggagatggcagaaccaagtacaacgaaaagttcaagggacgcgtgaccatgactagggatactagcacgaacaccgtgtacatggagctgtctagcctgacctccgaggacaccgctgtctacttttgcggccggggtggctacgaattcggaatcgattactggggccaggggacgaccgtgaccgtcagcagc

SEQ ID NO: 43 - DNA, 354 bp, synthetic construct
  source 1..354: mol_type = other DNA
caagttcagctggtgcagagcggcgcagaggtgaagaagcctggagcctctgttaaagtatcttgcaaagcatctggctatatctttacgaataatgatatgaattgggtccggcaggcccctggtcaaggccttgaatggatgggcgggatagatcccggcgatggtagaacgaaatacaacgaaaagtttaaaggacgagtgacgatgacgcgagacacttcaactaatacagtttatatggaactgtctagcctgacttctgaagatacagctgtatatttttgcggtcgaggggggtacgagttcggaattgattactggggacagggcacgaccgtaactgtaagtagc

SEQ ID NO: 44 - DNA, 63 bp, Homo sapiens
  source 1..63: mol_type = genomic DNA
atggccttaccagtgaccgccttgctcctgccgctggccttgctgctccacgccgccaggccg

SEQ ID NO: 45 - protein, 21 aa, Homo sapiens
MALPVTALLLPLALLLHAARP

SEQ ID NO: 46 - DNA, 45 bp, Homo sapiens
  source 1..45: mol_type = genomic DNA
ggtggcggtggctcgggcggtggtgggtcgggtggcggcggatct

SEQ ID NO: 47 - protein, 15 aa, synthetic construct
GGGGSGGGGSGGGGS

SEQ ID NO: 48 - DNA, 135 bp, Homo sapiens
  source 1..135: mol_type = genomic DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 49 - protein, 45 aa, Homo sapiens
TTTPAPRPPTPAPTIASQPLSLRPEACRPAAGGAVHTRGLDFACD

SEQ ID NO: 50 - DNA, 72 bp, Homo sapiens
  source 1..72: mol_type = genomic DNA
atctacatctgggcgcccttggccgggacttgtggggtccttctcctgtcactggttatcaccctttactgc

SEQ ID NO: 51 - protein, 24 aa, Homo sapiens
IYIWAPLAGTCGVLLLSLVITLYC

SEQ ID NO: 52 - DNA, 126 bp, Homo sapiens
  source 1..126: mol_type = genomic DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 53 - protein, 42 aa, Homo sapiens
KRGRKKLLYIFKQPFMRPVQTTQEEDGCSCRFPEEEEGGCEL

SEQ ID NO: 54 - DNA, 57 bp, Homo sapiens
  source 1..57: mol_type = genomic DNA
ctcgagcccaaatcttgtgacaaaactcacacatgcccaccgtgcccggatcccaaa

SEQ ID NO: 55 - protein, 19 aa, Homo sapiens
LEPKSCDKTHTCPPCPDPK

SEQ ID NO: 56 - DNA, 81 bp, Homo sapiens
  source 1..81: mol_type = genomic DNA
ttttgggtgctggtggtggttggtggagtcctggcttgctatagcttgctagtaacagtggcctttattattttctgggtg

SEQ ID NO: 57 - protein, 27 aa, Homo sapiens
FWVLVVVGGVLACYSLLVTVAFIIFWV

SEQ ID NO: 58 - DNA, 123 bp, Homo sapiens
  source 1..123: mol_type = genomic DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 59 - protein, 41 aa, Homo sapiens
RSKRSRLLHSDYMNMTPRRPGPTRKHYQPYAPPRDFAAYRS

SEQ ID NO: 60 - DNA, 336 bp, Homo sapiens
  source 1..336: mol_type = genomic DNA
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 61 - protein, 112 aa, Homo sapiens
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 15 aa, Homo sapiens
EPKSCDKTHTCPPCP

SEQ ID NO: 63 - protein, 15 aa, Homo sapiens
DKTHTCPPCPAPPVA

SEQ ID NO: 64 - protein, 12 aa, Homo sapiens
ESKYGPPCPPCP

SEQ ID NO: 65 - protein, 12 aa, Homo sapiens
ESKYGPPCPSCP

SEQ ID NO: 66 - protein, 49 aa, Homo sapiens
KPTTTPAPRPPTPAPTIASQPLSLRPEACRPAAGGAVHTRGLDFACDIY

SEQ ID NO: 67 - protein, 39 aa, Homo sapiens
IEVMYPPPYLDNERSNGTIIHVKGKHLCPSPLFPGPSKP

SEQ ID NO: 68 - protein, 47 aa, Homo sapiens
TTTPAPRPPTPAPTIASQPLSLRPEACRPAAGGAVHTRGLDFACDIY

SEQ ID NO: 69 - protein, 45 aa, Homo sapiens; identical to SEQ ID NO: 49

SEQ ID NO: 70 - protein, 21 aa, Homo sapiens
LCYLLDGILFIYGVILTALFL

SEQ ID NO: 71 - protein, 22 aa, Homo sapiens
MALIVLGGVAGLLLFIGLGIFF

SEQ ID NO: 72 - protein, 21 aa, Homo sapiens
IYIWAPLAGTCGVLLLSLVIT

SEQ ID NO: 73 - protein, 22 aa, Homo sapiens
IWAPLAGTCGVLLLSLVITLYC

SEQ ID NO: 74 - protein, 19 aa, Homo sapiens
IWAPLAGTCGVLLLSLVIT

SEQ ID NO: 75 - protein, 21 aa, Homo sapiens
FWLPIGCAAFVVVCILGCILI

SEQ ID NO: 76 - protein, 113 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 77 - protein, 42 aa, Homo sapiens; identical to SEQ ID NO: 53

SEQ ID NO: 78 - protein, 37 aa, Homo sapiens
KRGRKKLLYIFKQPFMRPVQTTQEEDGCSCRFPEEEE

SEQ ID NO: 79 - protein, 41 aa, Homo sapiens; identical to SEQ ID NO: 59

SEQ ID NO: 80 - protein, 42 aa, Homo sapiens
ALYLLRRDQRLPPDAHKPPGGGSFRTPIQEEQADAHSTLAKI

SEQ ID NO: 81 - protein, 38 aa, Homo sapiens
CWLTKKKYSSSVHDPNGEYMFMRAVNTAKKSRLTDVTL

SEQ ID NO: 82 - protein, 24 aa, Homo sapiens
LCARPRRSPAQEDGKVYINMPGRG

SEQ ID NO: 83 - protein, 52 aa, Homo sapiens
YFLGRLVPRGRGAAEAATRKQRITETESPYQELQGQRSDVYSDLNTQRPYYK

SEQ ID NO: 84 - protein, 48 aa, Homo sapiens
QRRKYRSNKGESPVEPAEPCHYSCPREEEGSTIPIQEDYRKPEPACSP

SEQ ID NO: 85 - protein, 5 aa, synthetic construct
NNDMN

SEQ ID NO: 86 - protein, 7 aa, synthetic construct
GYIFTNN

SEQ ID NO: 87 - protein, 9 aa, synthetic construct
GYIFTNNDM

SEQ ID NO: 88 - protein, 9 aa, synthetic construct
YIFTNNDMN

SEQ ID NO: 89 - protein, 8 aa, synthetic construct
YIFTNNDM

SEQ ID NO: 90 - protein, 7 aa, synthetic construct
YIFTNND

SEQ ID NO: 91 - protein, 8 aa, synthetic construct
IFTNNDMN

SEQ ID NO: 92 - protein, 7 aa, synthetic construct
IFTNNDM

SEQ ID NO: 93 - protein, 6 aa, synthetic construct
IFTNND

SEQ ID NO: 94 - protein, 7 aa, synthetic construct
FTNNDMN

SEQ ID NO: 95 - protein, 6 aa, synthetic construct
FTNNDM

SEQ ID NO: 96 - protein, 5 aa, synthetic construct
FTNND

SEQ ID NO: 97 - protein, 6 aa, synthetic construct
TNNDMN

SEQ ID NO: 98 - protein, 5 aa, synthetic construct
TNNDM

SEQ ID NO: 99 - protein, 4 aa, synthetic construct
TNND

SEQ ID NO: 100 - protein, 17 aa, synthetic construct
GIDPGDGRTKYNEKFKG

SEQ ID NO: 101 - protein, 8 aa, synthetic construct
IDPGDGRT

SEQ ID NO: 102 - protein, 9 aa, synthetic construct
IDPGDGRTK

SEQ ID NO: 103 - protein, 11 aa, synthetic construct
IDPGDGRTKYN

SEQ ID NO: 104 - protein, 9 aa, synthetic construct
GIDPGDGRT

SEQ ID NO: 105 - protein, 7 aa, synthetic construct
IDPGDGR

SEQ ID NO: 106 - protein, 10 aa, synthetic construct
DPGDGRTKYN

SEQ ID NO: 107 - protein, 9 aa, synthetic construct
DPGDGRTKY

SEQ ID NO: 108 - protein, 10 aa, synthetic construct
PGDGRTKYNE

SEQ ID NO: 109 - protein, 9 aa, synthetic construct
PGDGRTKYN

SEQ ID NO: 110 - protein, 13 aa, synthetic construct
GDGRTKYNEKFKG

SEQ ID NO: 111 - protein, 12 aa, synthetic construct
GDGRTKYNEKFK

SEQ ID NO: 112 - protein, 15 aa, synthetic construct
IDPGDGRTKYNEKFK

SEQ ID NO: 113 - protein, 13 aa, synthetic construct
DPGDGRTKYNEKF

SEQ ID NO: 114 - protein, 11 aa, synthetic construct
PGDGRTKYNEK

SEQ ID NO: 115 - protein, 9 aa, synthetic construct
GGYEFGIDY

SEQ ID NO: 116 - protein, 10 aa, synthetic construct
GRGGYEFGID

SEQ ID NO: 117 - protein, 9 aa, synthetic construct
GRGGYEFGI

SEQ ID NO: 118 - protein, 8 aa, synthetic construct
GRGGYEFG

SEQ ID NO: 119 - protein, 7 aa, synthetic construct
GRGGYEF

SEQ ID NO: 120 - protein, 10 aa, synthetic construct
RGGYEFGIDY

SEQ ID NO: 121 - protein, 8 aa, synthetic construct
RGGYEFGI

SEQ ID NO: 122 - protein, 9 aa, synthetic construct
RGGYEFGID

SEQ ID NO: 123 - protein, 8 aa, synthetic construct
GGYEFGID

SEQ ID NO: 124 - protein, 7 aa, synthetic construct
GGYEFGI

SEQ ID NO: 125 - protein, 8 aa, synthetic construct
GYEFGIDY

SEQ ID NO: 126 - protein, 7 aa, synthetic construct
GYEFGID

SEQ ID NO: 127 - protein, 7 aa, synthetic construct
YEFGIDY

SEQ ID NO: 128 - protein, 6 aa, synthetic construct
YEFGID

SEQ ID NO: 129 - protein, 17 aa, synthetic construct
KSSQSLLNSGNQKNSLA

SEQ ID NO: 130 - protein, 13 aa, synthetic construct
QSLLNSGNQKNSL

SEQ ID NO: 131 - protein, 14 aa, synthetic construct
QSLLNSGNQKNSLA

SEQ ID NO: 132 - protein, 13 aa, synthetic construct
SQSLLNSGNQKNS

SEQ ID NO: 133 - protein, 14 aa, synthetic construct
SQSLLNSGNQKNSL

SEQ ID NO: 134 - protein, 15 aa, synthetic construct
SQSLLNSGNQKNSLA

SEQ ID NO: 135 - protein, 14 aa, synthetic construct
SSQSLLNSGNQKNS

SEQ ID NO: 136 - protein, 15 aa, synthetic construct
SSQSLLNSGNQKNSL

SEQ ID NO: 137 - protein, 16 aa, synthetic construct
SSQSLLNSGNQKNSLA

SEQ ID NO: 138 - protein, 15 aa, synthetic construct
KSSQSLLNSGNQKNS

SEQ ID NO: 139 - protein, 16 aa, synthetic construct
KSSQSLLNSGNQKNSL

SEQ ID NO: 140 - protein, 13 aa, synthetic construct
SLLNSGNQKNSLA

SEQ ID NO: 141 - protein, 12 aa, synthetic construct
SLLNSGNQKNSL

SEQ ID NO: 142 - protein, 11 aa, synthetic construct
SLLNSGNQKNS

SEQ ID NO: 143 - protein, 7 aa, synthetic construct
GASTRES

SEQ ID NO: 144 - protein, 4 aa, synthetic construct
GAST

SEQ ID NO: 145 - protein, 5 aa, synthetic construct
GASTR

SEQ ID NO: 146 - protein, 6 aa, synthetic construct
GASTRE

SEQ ID NO: 147 - protein, 6 aa, synthetic construct
ASTRES

SEQ ID NO: 148 - protein, 5 aa, synthetic construct
ASTRE

SEQ ID NO: 149 - protein, 4 aa, synthetic construct
ASTR

SEQ ID NO: 150 - protein, 8 aa, synthetic construct
QNDHSFPL

SEQ ID NO: 151 - protein, 7 aa, synthetic construct
QNDHSFP

SEQ ID NO: 152 - protein, 8 aa, synthetic construct
NDHSFPLT

SEQ ID NO: 153 - protein, 7 aa, synthetic construct
NDHSFPL

SEQ ID NO: 154 - protein, 7 aa, synthetic construct
DHSFPLT

SEQ ID NO: 155 - protein, 6 aa, synthetic construct
DHSFPL

SEQ ID NO: 156 - DNA, 45 bp, synthetic construct; identical to SEQ ID NO: 46
  source 1..45: mol_type = other DNA

SEQ ID NO: 157 - protein, 10 aa, synthetic construct
GGGGSGGGGS

SEQ ID NO: 158 - protein, 20 aa, synthetic construct
GGGGSGGGGSGGGGSGGGGS

SEQ ID NO: 159 - protein, 25 aa, synthetic construct
GGGGSGGGGSGGGGSGGGGSGGGGS